(19) United States
(12) Patent Application Publication
(10) Pub. No.: US 2003/0134786 A1
(43) Pub. Date: Jul. 17, 2003

(54) TANGO 197 and TANGO 216 compositions and methods

(76) Inventors: James B. Rottman, Sudbury, MA (US); Theresa L. O'Keefe, Waltham, MA (US); Engin Ozkaynak, Milford, MA (US); Judith J. Healey, Newton, MA (US)

Correspondence Address: PENNIE & EDMONDS LLP, 1667 K Street, N.W., Washington, DC, 20006, US

(21) Appl. No.: 10/038,307

(22) Filed: Dec. 20, 2001

(51) Int. Cl. A61K 38/36; A61K 38/17

(52) U.S. Cl. 514/12

(57) ABSTRACT

The present application relates, in part, to methods and compositions for the prevention or amelioration of symptoms of anthrax. In particular, the present invention relates to TANGO 197 and/or TANGO 216 fusion polypeptides and their use as part of such methods.

### FIELD OF THE INVENTION

The present application relates, in part, to methods and compositions for the prevention or amelioration of symptoms of anthrax. In particular, the present invention relates to TANGO 197 and/or TANGO 216 fusion polypeptides and their use as part of such methods.

### BACKGROUND OF THE INVENTION

Anthrax, a disease, until recently, of primarily veterinary importance, is caused by the bacterium, Bacillus anthracis. This Gram positive, spore-forming bacterium is uniquely pathogenic because it produces a powerful exotoxin which allows the organism to evade clearance by the immune system. (Friedlander, A. M., et al. Curr Clin Top Infect Dis 20, 335-349 (2000)).

There are three forms of clinical disease seen in humans and the symptoms depend upon the route of exposure. Cutaneous exposure to Bacillus anthracis spores results in “wool sorters disease”, which is characterized by focal cutaneous gangrenous necrosis. In addition, inhalation or ingestion of anthrax spores cause the pulmonary and gastrointestinal forms of the disease. (Shafazand, S. et al. Chest 116, 1369-1376 (1999)). Treatment of affected individuals involves the administration of antibiotics and supportive care. Unfortunately, antibiotics kill the bacterium but do not neutralize the anthrax toxin, thus once clinical symptoms develop, the majority of cases are fatal. The spore-forming nature of the bacterium and ability to induce lethal disease in humans appear to be the main reasons that this organism has become a popular bioterror weapon. (Morgan, M. F. N J Med 97, 35-41 (2000)).

Anthrax spores enter the host by inhalation, ingestion or through an open skin wound. Following ingestion by phagocytic cells at the site of entry, the bacterium desporulates and begins to produce toxin. The toxin is composed of three components: the protective antigen (PA), edema factor (EF) and lethal factor (LF). PA binds to a specific anthrax receptor (ATR) on the surface of various host cells, and, after binding to the ATR, PA is cleaved into 2 fragments by a furin-like protease (Molloy, S. S. et al. J Biol Chem 267, 16396-16402 (1992)) located on the cell surface. The amino-terminal fragment PA₂₀ dissociates into the medium and this allows the carboxy-terminus PA₆₃ to heptamerize and bind LF and EF. (Milne, J. C. et al. J Biol Chem 269, 20607-20612 (1994))(Elliott, J. L., et al. Biochemistry 39, 6706-6713 (2000)). The toxin complex is subsequently internalized within an endocytic vesicle, and upon acidification of the endosomal environment, LF and EF are inserted into the cytoplasm of the target cell (Blaustein, R. O., et al. Proc Natl Acad Sci USA 86, 2209-2213 (1989))(Koehler, T. M. et al. Mol Microbiol 5, 1501-1506 (1991))(Milne, J. C., et al. Mol Microbiol 10, 647-653 (1993)). EF is an adenylate cyclase that has an inhibitory effect on phagocytic cells (Hoover, D. L. et al. Infect Immun 62, 4432-4439 (1994)) and LF is a protease that acts specifically to kill macrophages. (Duesbery, N. S. et al. Science 280, 734-737 (1998)).

The ATR has been reported to be a splice variant of a molecule known as TEM8 (Bradley, K. A. et al. Nature 414, 225-229 (2001); Carson-Walter, E. B. et al. Cancer Res 61, 6649-6655 (2001)). See also, WO 00/39149.

There have been various strategies to protect individuals from anthrax intoxication. The only effective protection currently available for humans is a vaccine that is composed of PA in adjuvant. (Pittman, P. R., et al. Vaccine 20, 972-978 (2001)) The disadvantage of this vaccine is that in order to achieve effective immunity, six injections must be given over 18 months and annual booster vaccinations are also required. Other methods currently being developed include vaccines composed of lethal factor in adjuvant or various DNA vaccination approaches. (Price, B. M. et al. Infect Immun 69, 4509-4515 (2001)) However, all of the aforementioned approaches require prophylactic immunization to achieve immunity and in order to effectively protect the U.S. population from an anthrax attack, 250 million individuals would have to be vaccinated.

Thus a great need exists for an effective prophylactic and therapeutic treatment for individuals at risk for or exposed to anthrax bacteria.

### SUMMARY OF THE INVENTION

In one aspect, the present invention provides a method for preventing a symptom of anthrax in a subject thought to be at risk for exposure to Bacillus anthracis, comprising: administering to the subject a pharmaceutically effective amount of a fusion polypeptide so that, if the subject is exposed to Bacillus anthracis, a symptom of said exposure is prevented, wherein said fusion polypeptide comprises a von Willebrand factor A-like domain (vWF) amino acid sequence and an amino acid sequence heterologous to said vWF.

In another aspect, the present invention provides a method for preventing a symptom of anthrax in a subject suspected of having been exposed to Bacillus anthracis, comprising: administering to the subject a pharmaceutically effective amount of a fusion polypeptide so that, if the subject has been exposed to Bacillus anthracis, a symptom of said exposure is prevented, wherein said fusion polypeptide comprises a von Willebrand factor A-like domain (vWF) amino acid sequence and an amino acid sequence heterologous to said vWF.

In yet another aspect, the present invention provides a method for ameliorating a symptom of anthrax in a subject in need of said amelioration, comprising: administering to the subject a pharmaceutically effective amount of a fusion polypeptide so that a symptom of anthrax is ameliorated, wherein said fusion polypeptide comprises a von Willebrand factor A-like domain (vWF) amino acid sequence and an amino acid sequence heterologous to said vWF.

The methods of the present invention can, for example, be utilized to prevent or ameliorate a symptom of cutaneous and/or inhalation anthrax.

With respect to these methods, the vWF can, for example, be a TANGO 197 vWF as depicted in SEQ ID NO:2. For example, the TANGO 197 vWF can comprise amino acid residues 44-215, 44-216, 29-316, 29-318, or 29-333 of the amino acid sequence depicted in SEQ ID NO:2.

The vWF of the fusion polypeptides utilized as part of the invention can also, for example, be a TANGO 216 vWF as depicted in SEQ ID NO:6. For example, the TANGO 216 vWF can comprise amino acid residues 44-213 of the amino acid sequence depicted in SEQ ID NO:6.

Alternatively, the fusion polypeptide utilized as part of these methods can comprise amino acid residues 29-316, 29-318, or 29-333 of the amino acid sequence depicted in SEQ ID NO:2, except that the amino acid sequence differs from said amino acid residues 29-316, 29-318, or 29-333 of SEQ ID NO:2 in that a cysteine residue of the amino acid sequence has been converted to another amino acid residue. In preferred embodiments, the converted amino acid residue is not within the vWF amino acid sequence.

The fusion polypeptides utilized as part of these methods can be such that the heterologous amino acid sequence comprises a human immunoglobulin constant region, such as a human IgG1 constant region, including a modified human IgG1 constant region wherein the IgG1 constant region does not bind Fc receptor and/or does not initiate antibody-dependent cellular cytotoxicity (ADCC) reactions.

The heterologous amino acid sequence of the fusion polypeptides utilized as part of the present invention can also comprise an amino acid sequence useful for identifying, tracking or purifying the fusion polypeptide, e.g., can comprise a FLAG or a His tag sequence. The fusion polypeptide can further comprise an amino acid sequence containing a proteolytic cleavage site which can, for example, be useful for removing the heterologous amino acid sequence from the TANGO 197 or TANGO 216 sequence of the fusion polypeptide.

Among the particular fusion polypeptides that can be utilized as part of the invention are, for example, fusion polyeptides that comprises amino acid residues 29-549 of pLKTOK125 (SEQ ID NO:10), 29-540 of pLKTOK126 (SEQ ID NO:12), 29-549 of pLKTOK127 (SEQ ID NO:14), 29-549 of pLKTOK129 (SEQ ID NO:16), 29-551 of pO610 (SEQ ID NO:18), 29-564 of pO611 (SEQ ID NO:20), 29-342 of pO613 (SEQ ID NO:22), 29-345 of pO614 (SEQ ID NO:24), or 29-327 of pO615 (SEQ ID NO:26).

The present invention also provides fusion polypeptides comprising a von Willebrand factor A-like domain (vWF) amino acid sequence and an amino acid sequence heterologous to said vWF. The vWF can, for example, be a TANGO 197 vWF as depicted in SEQ ID NO:2. For example, the TANGO 197 vWF can comprise amino acid residues 44-215, 44-216, 29-316, 29-318, or 29-333 of the amino acid sequence depicted in SEQ ID NO:2.

The present invention also provides fusion polypeptides comprising a von Willebrand factor A-like domain (vWF) amino acid sequence and an amino acid sequence heterologous to said vWF. The vWF can, for example, be a TANGO 197 vWF as depicted in SEQ ID NO:4. For example, the TANGO 197 vWF can comprise amino acid residues 44-215, 44-216, 29-316, 29-318, or 29-333 of the amino acid sequence depicted in SEQ ID NO:2.

The present invention also provides fusion polypeptides comprising a von Willebrand factor A-like domain (vWF) amino acid sequence and an amino acid sequence heterologous to said vWF. The vWF can, for example, be a TANGO 216 vWF as depicted in SEQ ID NO:6. For example, the TANGO 216 vWF can comprise amino acid residues 44-213, 29-316, 29-318, or 29-333 of the amino acid sequence depicted in SEQ ID NO:6. The vWF can also, for example, be a TANGO 216 vWF as depicted in SEQ ID NO:8.

Alternatively, the fusion polypeptide of the invention can comprise amino acid residues 29-316, 29-318, or 29-333 of the amino acid sequence depicted in SEQ ID NO:2, except that the amino acid sequence differs from said amino acid residues 29-316, 29-318, or 29-333 of SEQ ID NO:2 in that a cysteine residue of the amino acid sequence has been converted to another amino acid residue. In preferred embodiments, the converted amino acid residue is not within the vWF amino acid sequence.

The fusion polypeptides of the invention can be such that the heterologous amino acid sequence comprises a human immunoglobulin constant region, such as a human IgG1 constant region, including a modified human IgG1 constant region wherein the IgG1 constant region does not bind Fc receptor and/or does not initiate antibody-dependent cellular cytotoxicity (ADCC) reactions.

The heterologous amino acid sequence of the fusion polypeptides utilized as part of the present invention can also comprise an amino acid sequence useful for identifying, tracking or purifying the fusion polypeptide, e.g., can comprise a FLAG or a His tag sequence. The fusion polypeptide can further comprise an amino acid sequence containing a proteolytic cleavage site which can, for example, be useful for removing the heterologous amino acid sequence from the TANGO 197 or TANGO 216 sequence of the fusion polypeptide.

Among the particular fusion polypeptides of the invention are, for example, fusion polyeptides that comprises the amino acid sequence of pLKTOK125 (SEQ ID NO:10), pLKTOK126 (SEQ ID NO:12), pLKTOK127 (SEQ ID NO:14), pLKTOK129 (SEQ ID NO:16), pO610 (SEQ ID NO:18), pO611 (SEQ ID NO:20), pO613 (SEQ ID NO:22), pO614 (SEQ ID NO: 24), or pO615 (SEQ ID NO:26). More preferably, among the particular fusion polypeptides of the invention are, for example, fusion polyeptides comprising amino acid residues 29-549 of pLKTOK125 (SEQ ID NO:10), 29-540 of pLKTOK126 (SEQ ID NO:12), 29-549 of pLKTOK127 (SEQ ID NO:14), 29-549 of pLKTOK129 (SEQ ID NO:16), 29-551 of pO610 (SEQ ID NO:18), 29-564 of pO611 (SEQ ID NO:20), 29-342 of pO613 (SEQ ID NO:22), 29-345 of pO614 (SEQ ID NO:24), or 29-327 of pO615 (SEQ ID NO:26).

The present invention also provides compositions that comprise a fusion polypeptide of the invention and a pharmaceutically acceptable carrier, excipient or diluent.

### BRIEF DESCRIPTION OF THE DRAWINGS

FIG. 1 depicts the cDNA sequence of human TANGO 197 and predicted amino acid sequence of TANGO 197 (SEQ ID NO: 2). The open reading frame extends from nucleotide 213 to 1211 of SEQ ID NO:1.

FIG. 2 depicts the alignment of amino acids 44 to 215 of TANGO 197 and the von Willebrand Factor (vWF) consensus sequence. In these alignments, an uppercase letter between the two sequences indicates an exact match, and a (+) indicates a conservative amino acid substitution.

FIG. 3 depicts the cDNA sequence of mouse TANGO 197 and predicted amino acid sequence of mouse TANGO 197 (SEQ ID NO: 4). The open reading frame extends from nucleotide 3 to 1145 of SEQ ID NO: 3.

FIG. 4 depicts diagramatically the similarities between the mouse TANGO 197 and Human TANGO 197.

FIG. 5 depicts the cDNA sequence of human TANGO 216 and predicted amino acid sequence of human TANGO 216 (SEQ ID NO: 6). The open reading frame extends from nucleotide 307 to 1770 of SEQ ID NO: 5.

FIG. 6 depicts the cDNA sequence of mouse TANGO 216 and predicted amino acid sequence of mouse TANGO 216 (SEQ ID NO: 8). The open reading frame extends from nucleotide 149 to 1609 of SEQ ID NO:7.

FIG. 7 depicts the alignment of the amino acid sequence of human TANGO 216 and mouse TANGO 216. In this alignment, a (|) between the two sequences indicates an exact match and a (:) indicates similarity.

FIG. 8 depicts the alignment of the amino acid sequence of human TANGO 197 and human TANGO 216. In this alignment, a (|) between the two sequences indicates an exact match and a (:) indicates similarity.

FIG. 9 depicts the alignment of the amino acid sequence of mouse TANGO 197 and mouse TANGO 216. In this alignment, a (|) between the two sequences indicates an exact match and a (:) indicates similarity.

FIG. 10 depicts a diagrammatic representation of the various constructs for plasmids pO610, based upon mouse TANGO 197 amino acid sequence, and plasmids pO612, pO611, pO613, pO614, and pO615, based upon human TANGO 197 amino acid sequence.

FIG. 11 depicts the nucleic acid sequence (SEQ ID NO:17) and amino acid sequence (SEQ ID NO:18) of the mouse TANGO 197 Ig fusion protein produced by plasmid pO610.

FIG. 12 depicts the nucleic acid sequence (SEQ ID NO:19) and amino acid sequence (SEQ ID NO:20) of the human TANGO 197 Ig fusion protein produced by plasmid pO611.

FIG. 13 depicts the nucleic acid sequence (SEQ ID NO:21) and amino acid sequence (SEQ ID NO:22) of the human TANGO 197 FLAG fusion protein produced by plasmid pO613.

FIG. 14 depicts the nucleic acid sequence (SEQ ID NO:23) and amino acid sequence (SEQ ID NO:24) of the human TANGO 197 HisTag fusion protein produced by plasmid pO614.

FIG. 15 depicts the nucleic acid sequence (SEQ ID NO:25) and amino acid sequence (SEQ ID NO:26) of the human TANGO 197 protein produced by plasmid pO615.

FIG. 16 depicts a diagramatic representation of the various constructs for plasmids pLKTOK125, pLKTOK126, pLKTOK127, and pLKTOK129 based upon human TANGO 197 amino acid sequence.

FIG. 17 depicts the signal peptide-containing human TANGO 197 Ig FcR mutated fusion nucleic acid sequence (SEQ ID NO:9) and the mature human TANGO 197 Ig FcR mutated fusion protein amino acid sequence (SEQ ID NO:10) produced by plasmid pLKTOK125 in which the cysteine residue at amino acid position 317 is mutated to serine.

FIG. 18 depicts the immunoglobulin leader-containing human TANGO 197 Ig FcR mutated fusion nucleic acid sequence (SEQ ID NO:11) and the mature human TANGO 197 Ig FcR mutated fusion protein amino acid sequence (SEQ ID NO:12) produced by plasmid pLKTOK126 in which the cysteine residue at amino acid position 317 is mutated to serine.

FIG. 19 depicts the signal peptide-containing human TANGO 197 Ig FcR mutated fusion nucleic acid sequence (SEQ ID NO:13) and the mature human TANGO 197 Ig FcR mutated fusion protein amino acid sequence (SEQ ID NO:14) produced by plasmid pLKTOK127 with a cysteine residue at amino acid position 317.

FIG. 20 depicts the signal peptide-containing human TANGO 197 Ig wild-type FcR fusion nucleic acid sequence (SEQ ID NO:15) and the mature human TANGO 197 Ig wild-type FcR fusion protein amino acid sequence (SEQ ID NO:16) produced by plasmid pLKTOK129 with a cysteine residue at amino acid position 317.

### DETAILED DESCRIPTION OF THE INVENTION

TANGO 197 and TANGO 216 Fusion Polypeptides

It is noted that, unless otherwise stated, as used herein, the terms “protein,” “polypeptide,” and “peptide,” are interchangeable. Further, unless otherwise stated, the TANGO 197 and/or TANGO 216 amino acid sequences of the fusion polypeptides of the invention are present within the fusion polypeptides in an operably-linked configuration. That is, within the fusion protein, the TANGO 197 and/or TANGO 216 amino acid residues and the heterologous amino acid sequences are fused in-frame to each other. The heterologous amino acid sequences can be fused to the N-terminal to, C-terminal to, or within TANGO 197 and/or TANGO 216 sequences. In addition, unless otherwise stated, as used herein, the terms “signal peptide,” “signal sequence,” and “leader sequence” are considered interchangeable.

The present invention relates, in part, to fusion polypeptides comprising TANGO 197 and/or TANGO 216 sequences. Briefly, any of the TANGO 197 and/or TANGO 216 polypeptides described herein, including, but not limited to TANGO 197 and/or TANGO 216, fragments, derivatives, naturally occurring allelic variants, or biologically active portions thereof, can be operably linked to a heterologous amino acid sequence or sequences to form fusion proteins. A heterologous amino acid sequence can be fused to the N-terminus, the C-terminus, and/or located within the amino acid sequence of the TANGO 197 and/or TANGO 216 polypeptide of the invention.

In general, a fusion polypeptide of the invention comprises, in its mature form, a TANGO 197 and/or TANGO 216 amino acid sequence and one or more sequences heterologous to TANGO 197 and TANGO 216. The immature form of a fusion polypeptide of the invention, in general, further comprise a signal peptide, either native or heterologous to the TANGO sequence or sequences being utilized as part of the fusion polypeptide.

In one embodiment of such a fusion polypeptide, the fusion polypeptide comprises a TANGO 197 amino acid sequence, preferably a human TANGO 197 amino acid sequence. In another embodiment, the fusion polypeptide comprises a TANGO 216 amino acid sequence, preferably a human TANGO 216 amino acid sequence. In yet another embodiment, the fusion polypeptide comprises a TANGO 197 amino acid sequence, preferably a human TANGO 197 amino acid sequence, and a TANGO 216 amino acid sequence, preferably a human TANGO 216 amino acid sequence.

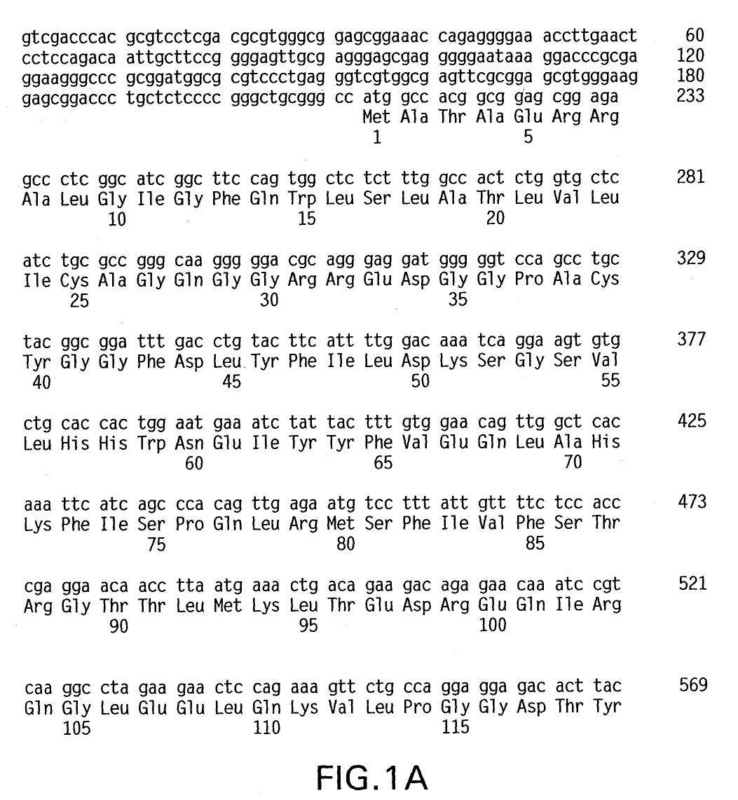

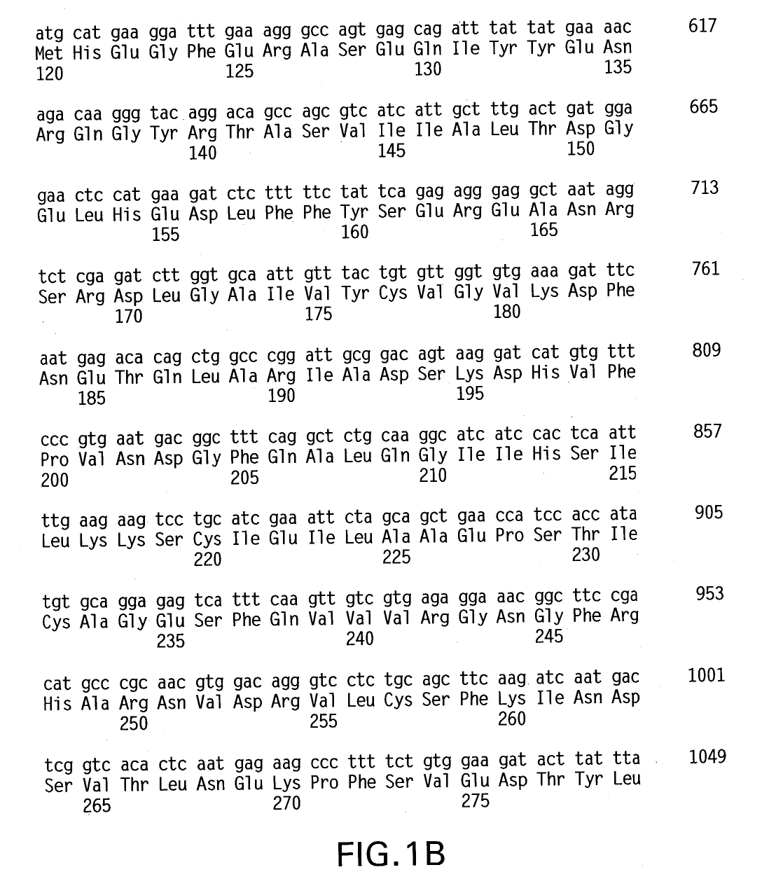

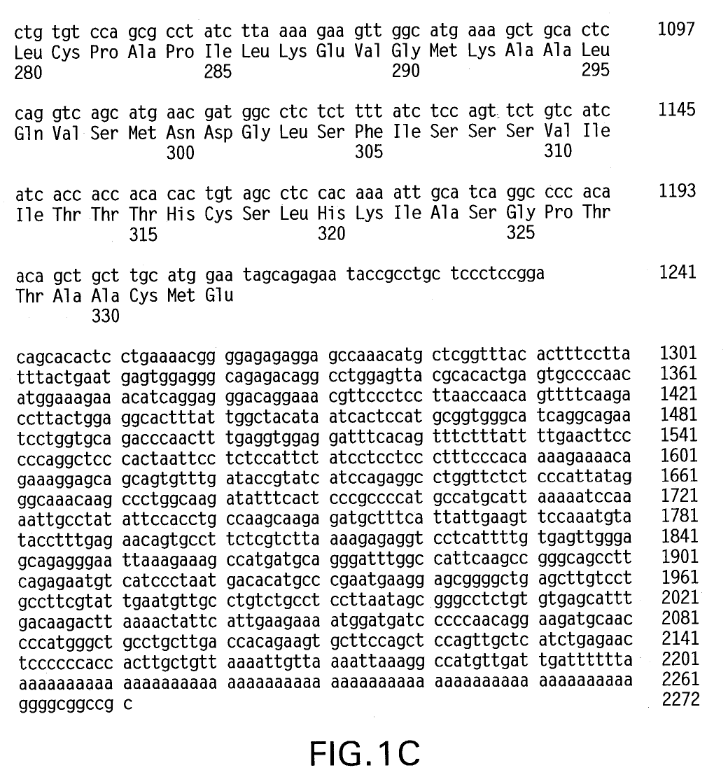

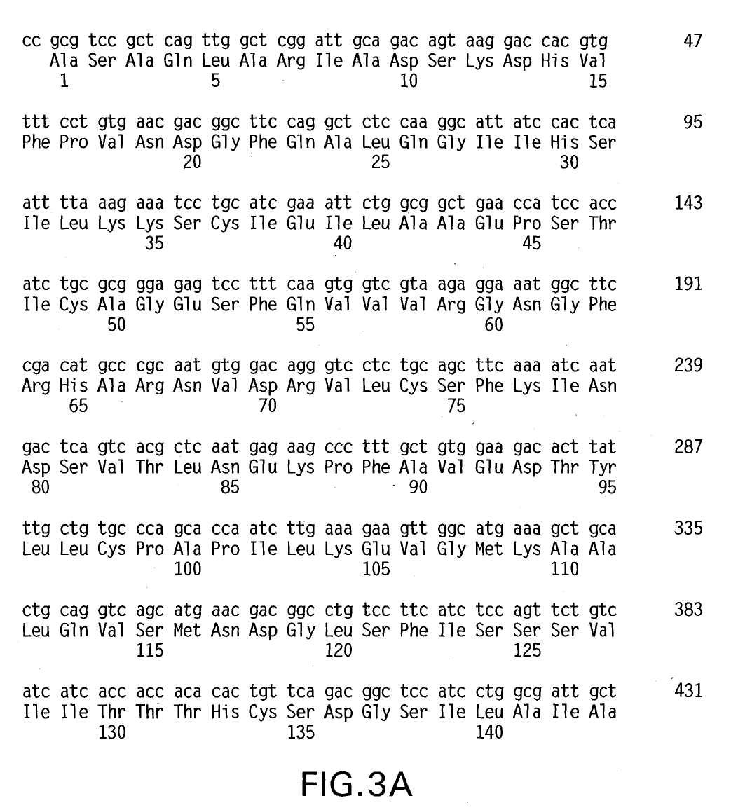

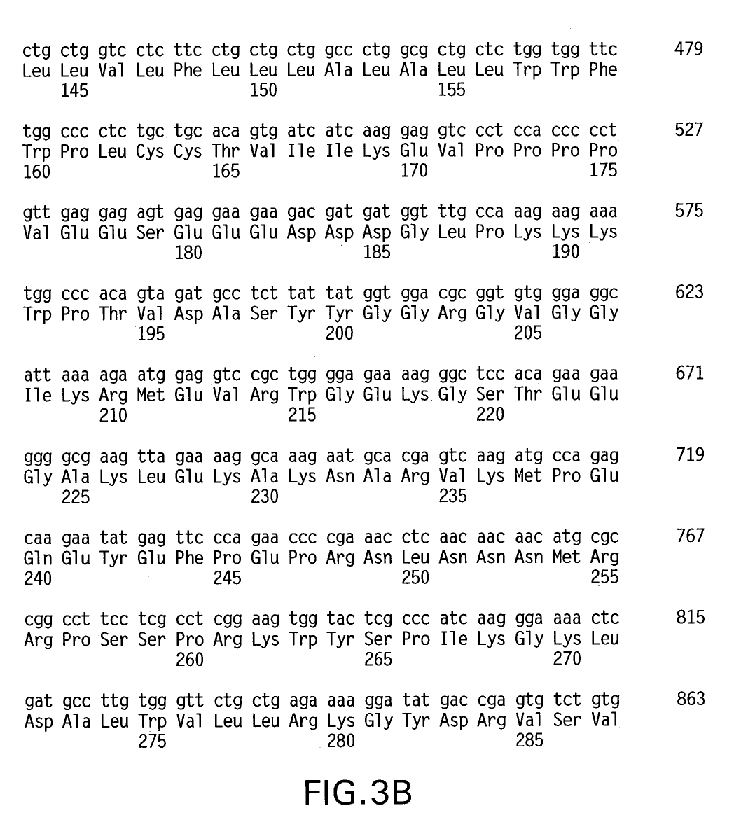

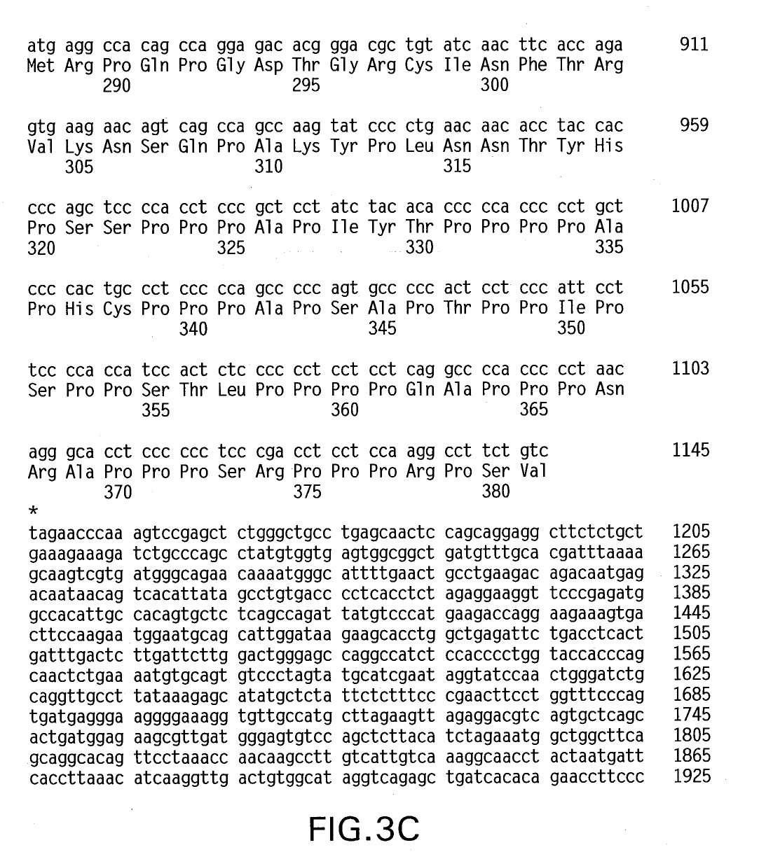

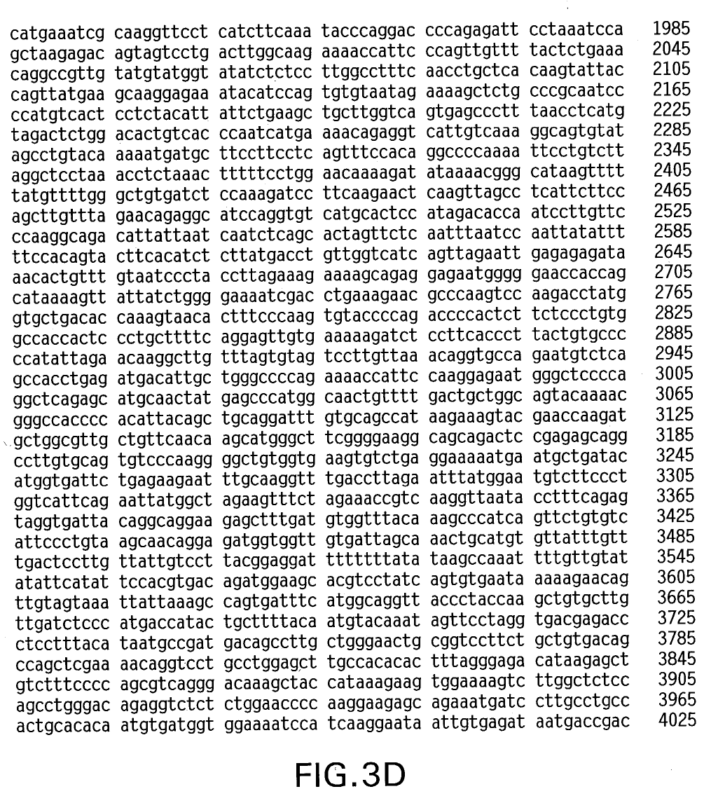

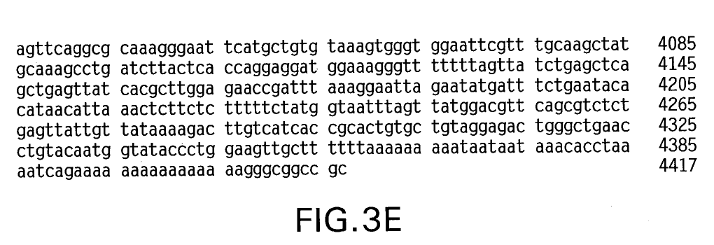

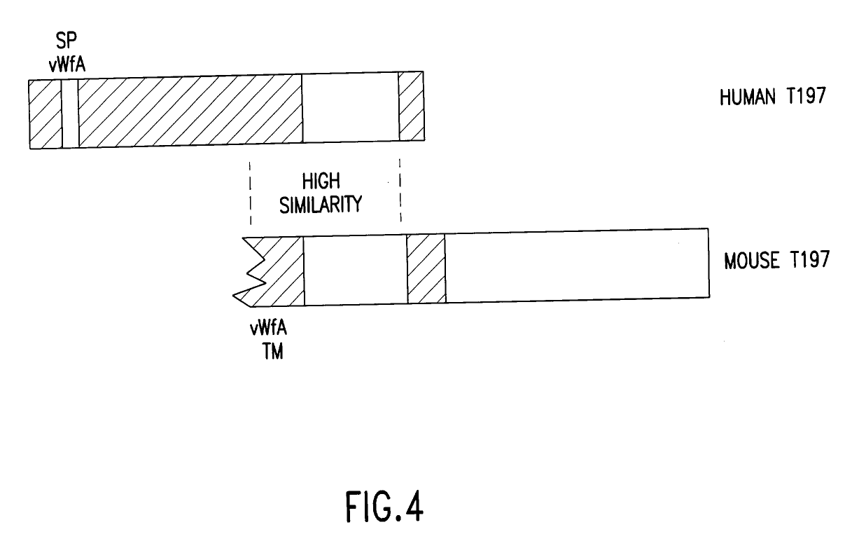

As described above, the fusion polypeptides of the invention can comprise any of the TANGO 197 amino acid sequences discussed herein. In one embodiment, a fusion polypeptide of the invention comprises a portion of a human TANGO 197 amino acid sequence, for example a portion of the amino acid sequence depicted in FIG. 1 (SEQ ID NO:2), or a portion of the amino acid sequence depicted in FIG. 3 (SEQ ID NO:4). Representative examples of such fusion polypeptides are presented in the Example, below.

Such a fusion polypeptide of the invention can, e.g., comprise a von Willebrand factor domain A-like region (vWF). As used herein, a vWF domain refers to an amino acid sequence of about 150 to 200, preferably about 160 to 190, 170 to 180, and more preferably about 172 to 175 amino acids in length. Thus, in one embodiment, A vWF domain of human TANGO 197 extends, for example, from about amino acids 44 to 215 or 44 to 216 of SEQ ID NO:2, and a fusion polypeptide of the invention comprises amino acid residues 44-215 or 44 to 216 of SEQ ID NO:2. In another embodiment, a vWF domain of TANGO 197 extends from about amino acids 44 to 215 or 44 to 216 of SEQ ID NO:4, and a fusion polypeptide of the invention comprises amino acid residues 44 to 215 or 44 to 216 of SEQ ID NO:4.

Such fusion polypeptides can further comprise additional amino acid residues of a TANGO 197 extracellular or secreted domain, e.g., a human TANGO 197 extracellular or secreted domain, up to full-length extracellular domain, for example amino acid residues 26, 27, 28, 29, or 30 to 301, 316, 318, 319, 320, or 333 of FIG. 1 (SEQ ID NO:2) or amino acid residues 26, 27, 28, 29, or 30 to 301, 316, 318, 319, 320, or 333, or, for example amino acid residues 26, 27, 28, 29, or 30 to 130, 131, 132, 135, 134, or 135 of FIG. 3 (SEQ ID NO:4). Extracellular domain amino acid residues utilized as part of the fusion polypeptides can include, for example, full-length extracellular domains lacking, for example, one, two, three, four, five, or six amino acid residues just amino to the transmembrane domain. In addition, the extracellular domain amino acid residues can differ from the wild type TANGO 197 amino acid sequence.

For example, one or more of the cysteine residues of the TANGO 197 sequence utilized as part of the fusion polypeptide can be converted to another residue, for example, to avoid intermolecular disulfide bonding interactions. In particular, the existence of certain cysteine amino acid residues capable of forming unwanted intermolecular disulfide bridges within or between each stated component of the TANGO 197 or TANGO 216 fusion protein may be advantageously modified so as to eliminate or reduce such unwanted interactions. Unwanted cysteine amino acids may be mutated by way of a conservative amino acid substitution. As used herein, a “conservative amino acid substitution” is one in which the amino acid residue is replaced with an amino acid residue having a similar side chain. Families of amino acid residues having similar side chains have been defined in the art. In case of the amino acid cysteine, amino acids with uncharged polar side chains (e.g., glycine, serine, threonine, tyrosine) as well as alanine represent preferable replacement amino acid residues.

Such a fusion polypeptide can also further comprise a TANGO 197 transmembrane domain, for example amino acid residues 302, 319, 320, 321, or 322 to 341, 342, 343, or 344 of FIG. 1 (SEQ ID NO:2). In another embodiment, a fusion polypeptide of the invention can also, for example, comprise a TANGO 197 transmembrane domain of amino acids 129, 130, 131, 132, or 133 to 160, 161, 162, 163, or 164 of FIG. 3 (SEQ ID NO:4). In still another embodiment, a fusion polypeptide of the invention can further comprise amino acid residues of a TANGO 197 intracellular domain, for example, amino acid residues 341, 342, 343, or 344 to 381 of FIG. 3 (SEQ ID NO:4).

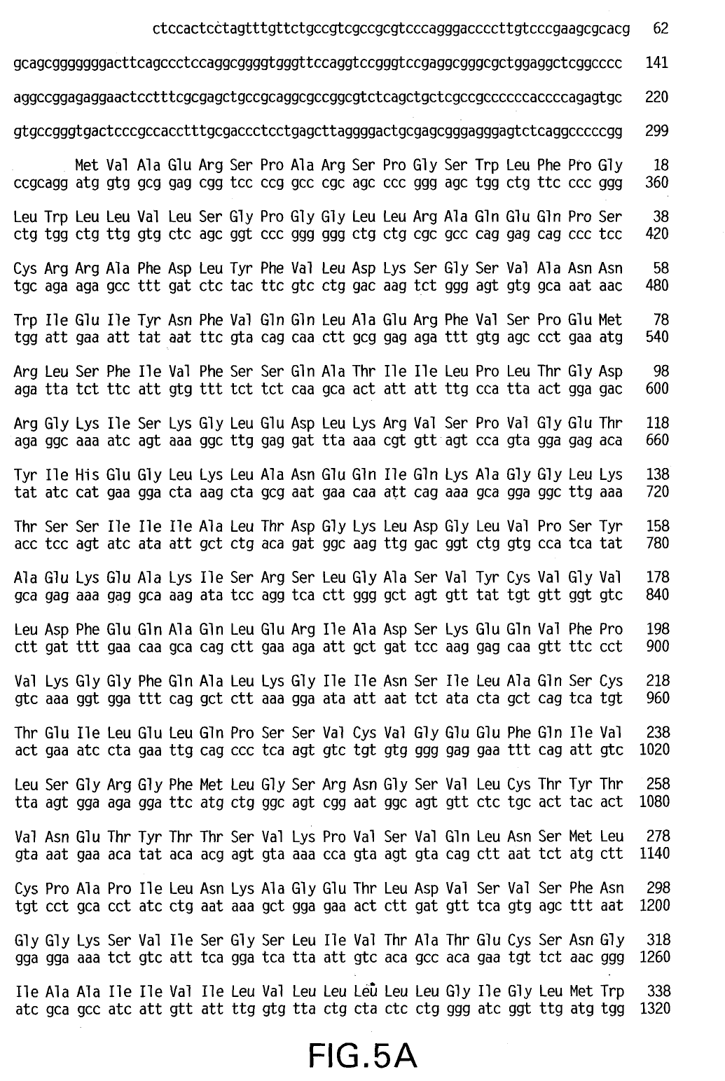

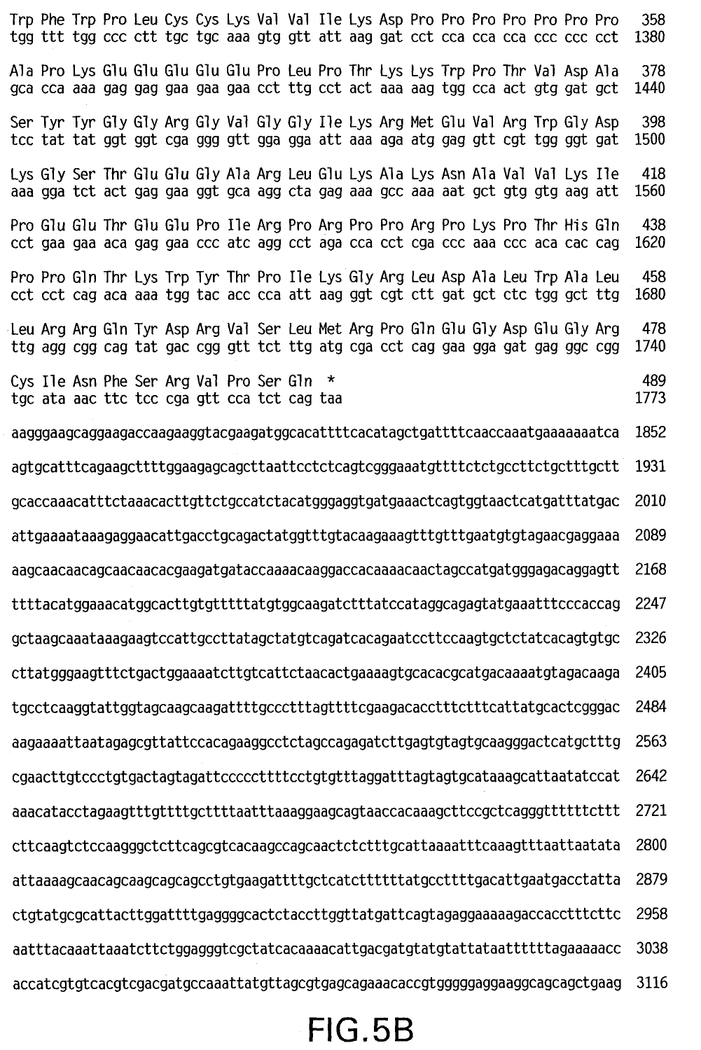

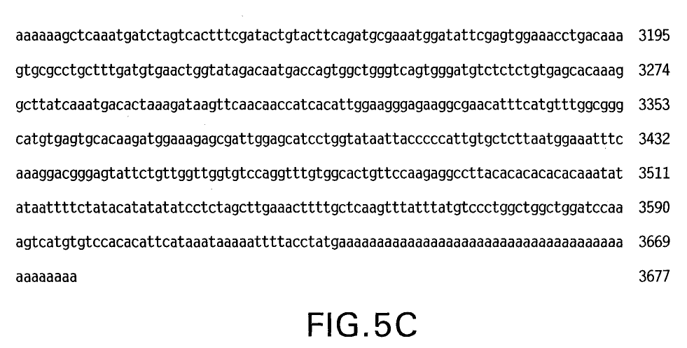

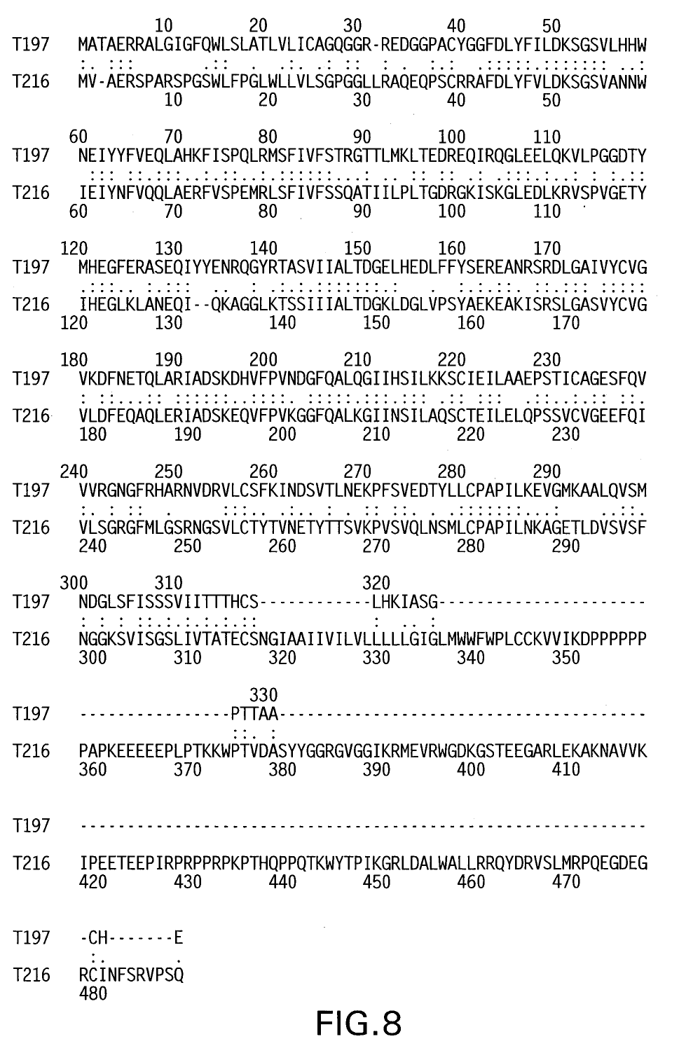

As described above, the fusion polypeptides of the invention can comprise any of the TANGO 216 amino acid sequences discussed herein. In one embodiment, a fusion polypeptide of the invention comprises a portion of a human TANGO 216 amino acid sequence, for example a portion of the amino acid sequence depicted in FIG. 5 (SEQ ID NO:6), or a portion of the amino acid sequence depicted in FIG. 8 (SEQ ID NO:8).

Such a fusion polypeptide of the invention can, e.g., comprise a von Willebrand factor domain A-like region (vWF). In one embodiment, a vWF domain of human TANGO 216 extends, for example, from about amino acids 44 to 213, 214, 215 or 216 of SEQ ID NO:6, and a fusion polypeptide of the invention comprises amino acid residues 44 to 213, 214, 215 or 216 of SEQ ID NO:6. In another embodiment, a vWF domain of TANGO 216 extends from about amino acids 44 to 213, 214, 215 or 216 of SEQ ID NO:8, and a fusion polypeptide of the invention comprises amino acid residues 44 to 213, 214, 215 or 216 of SEQ ID NO:8.

Such fusion polypeptides can further comprise additional amino acid residues of a TANGO 216 extracellular domain, e.g., a human TANGO 216 extracellular domain, up to a full-length extracellular domain, for example amino acid residues 34-79 and 342-488 of FIG. 5 (SEQ ID NO:6) or amino acid residues 34-79 and 342-487 of FIG. 8 (SEQ ID NO:8). Extracellular domain amino acid residues utilized as part of the fusion polypeptides can include, for example, full-length extracellular domains lacking, for example, one, two, three, four, five, or six amino acid residues just amino to the transmembrane domain. In addition, the extracellular domain amino acid residues can differ from the wild type TANGO 216 amino acid sequence. For example, one or more of the cysteine residues of the TANGO 216 sequence utilized as part of the fusion polypeptide can be mutated to another residue, for example, to avoid intermolecular disulfide bonding interactions, as discussed above.

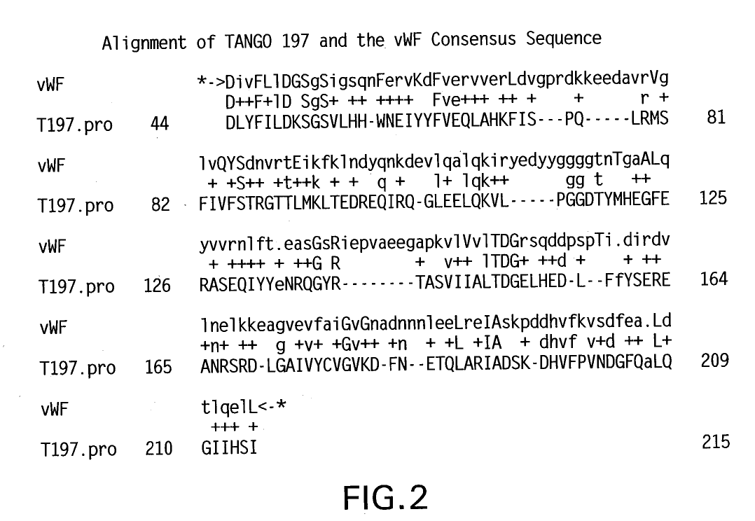

Such a fusion polypeptide can also further comprise a TANGO 216 transmembrane domain, e.g., a human TANGO 216 transmembrane domain, for example amino acid residues 80-97 and 318-341 of FIG. 5 (SEQ ID NO:6). In another embodiment, a fusion polypeptide of the invention can also, for example, comprise a TANGO 216 transmembrane domain, for example, amino acid residues 80-97 and 318 to 341 of FIG. 8 (SEQ ID NO:8). In still another embodiment, a fusion polypeptide of the invention can further comprise amino acid residues of a TANGO 216 intracellular domain, for example, amino acid residues 98 to 317 of FIG. 1 (SEQ ID NO:2), or amino acid residues 97 to 318 of FIG. 2 (SEQ ID NO:4).

As also described above, the fusion polypeptides can comprise a TANGO 197 and a TANGO 216 amino acid sequence, such as one or more of the amino acid sequences described above.

The fusion polypeptides of the invention also comprise sequences heterologous to TANGO 197 and/or TANGO 216. In one embodiment, the TANGO 197 and/or TANGO 216 fusion protein comprises a heterologous sequence that is a sequence derived from a member of the immunoglobulin protein family, for example, comprise an immunoglobulin constant region, e.g., a human immunoglobulin constant region such as a human IgG1 constant region. The fusion protein can, for example, comprise a portion of a TANGO 197 and/or TANGO 216 polypeptide fused with the amino-terminus or the carboxyl-terminus of an immunoglobulin constant region, as disclosed, e.g., in U.S. Pat. No. 5,714,147, U.S. Pat. No. 5,116,964, U.S. Pat. No. 5,514,582, and U.S. Pat. No. 5,455,165. In those embodiments in which all or part of a polypeptide of the invention is fused with sequences derived from a member of the immunoglobulin protein family, the FcR region of the immunoglobulin may be either wild-type or mutated. In certain embodiments, it is desirable to utilize an immunoglobulin fusion protein that does not inteact with a Fc receptor and does not initiate ADCC reactions. In such instances, the immunoglobulin heterologous sequence of the fusion protein can be mutated to inhibit such reactions. See, e.g., U.S. Pat. No. 5,985,279 and WO 98/06248.

In another embodiment, heterologous amino acid sequence of the fusion polypeptides of the present invention can also comprise an amino acid sequence useful for identifying, tracking or purifying the fusion polypeptide, e.g., can comprise a FLAG (see, e.g., Hoop, T. P. et al., Bio/Technology 6, 1204-1210 (1988); Prickett, K. S. et al., BioTechniques 7, 580-589 (1989)) or a His tag (Van Reeth, T. et al., BioTechniques 25, 898-904 (1998)) sequence. The fusion polypeptide can further comprise an amino acid sequence containing a proteolytic cleavage site which can, for example, be useful for removing the heterologous amino acid sequence from the TANGO 197 or TANGO 216 sequence of the fusion polypeptide.

In another embodiment, the TANGO 197 and/or TANGO 216 fusion protein comprises a GST fusion protein in which the polypeptide of the invention is fused to the C-terminus of GST sequences. Such fusion protein can facilitate the purification of a recombinant polypeptide of the invention.

In those embodiments in which a GST, FLAG or HisTag fusion constructs is employed in the construction of the TANGO 197 and/or TANGO 216 fusion proteins, proteolytic cleavage sites may be optionally introduced at the junction of the fusion moiety and the TANGO 197 and/or TANGO 216 protein to enable separation of the TANGO 197 and/or TANGO 216 protein from the fusion moiety subsequent to purification of the TANGO 197 and/or TANGO 216 fusion protein. Such enzymes, and their cognate recognition sequences, include, for example, without limitation, Factor Xa, thrombin and enterokinase. Typical fusion expression vectors include pGEX (Pharmacia Biotech Inc.; Smith and Johnson (1988) Gene 67:31-40), pMAL (New England Biolabs, Beverly, Mass.) and pRIT5 (Pharmacia, Piscataway, N.J.) which may be used to fuse glutathione S-transferase (GST), maltose E binding protein, or protein A, respectively, to the target TANGO 197 and/or TANGO 216 protein.

The fusion polypeptides of the invention will generally be expressed as immature forms comprising an amino-terminal signal peptide that can be cleaved off. Such signal peptides are well known. In one embodiment, a fusion polypeptide of the invention comprises a signal sequence of amino acids 1 to 25, 1 to 26, 1 to 27, 1 to 28, or 1 to 29, of SEQ ID NO:2. In another embodiment, a fusion polypeptide of the invention comprises a signal sequence of amino acids 1 to 25, 1 to 26, 1 to 27, 1 to 28, or 1 to 29 of SEQ ID NO:4. In another embodiment, a fusion polypeptide of the invention comprises a signal sequence of amino acids 1 to 31, 1 to 32, 1 to 33, 1 to 34 or 1 to 35 of SEQ ID NO:6. In another embodiment, a fusion polypeptide of the invention comprises a signal sequence of amino acids 1 to 31, 1 to 32, 1 to 33, 1 to 34 or 1 to 35 of SEQ ID NO:8.

In other embodiments, the fusion protein contains a signal sequence heterologous to TANGO 197 and TANGO 216. Signal sequences generally include a peptide of at least about 15 or 20 amino acid residues in length which occurs at the N-terminus of secretory and membrane-bound proteins and which contains at least about 70% hydrophobic amino acid residues such as alanine, leucine, isoleucine, phenylalanine, proline, tyrosine, tryptophan, or valine. In a preferred embodiment, a signal sequence contains at least about 10 to 40 amino acid residues, preferably about 19-34 amino acid residues, and has at least about 60-80%, more preferably at least about 65-75%, and more preferably at least about 70% hydrophobic residues. A signal sequence serves to direct a protein containing such a sequence to a lipid bilayer. A signal sequence is usually cleaved during processing of the mature protein.

For example, the gp67 secretory sequence of the baculovirus envelope protein can be used as a heterologous signal sequence (Current Protocols in Molecular Biology, Ausubel et al., eds., John Wiley & Sons, 1992). In addition, a human Ig signal peptide, e.g., a human IgG1 signal peptide can be used. See, e.g., U.S. patent application filed Oct. 19, 2001, identified as internal reference number MPI2001-244P1(M). Other examples of eukaryotic heterologous signal sequences include, but are not limited to, the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

The fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Ausubel et al., supra). Moreover, many expression vectors are commercially available that already encode a fusion moiety (e.g., a GST polypeptide). A nucleic acid encoding a tango 197 and/or TANGO 216 sequence of interest can be cloned into such an expression vector such that the fusion moiety is linked in-frame to the polypeptide of the invention.

In another embodiment, the signal sequences of the present invention can be used to identify regulatory sequences, e.g., promoters, enhancers, repressors. Since signal sequences are the most amino-terminal sequences of a peptide, it is expected that the nucleic acids which flank the signal sequence on its amino-terminal side will be regulatory sequences which affect transcription. Thus, a nucleotide sequence which encodes all or a portion of a signal sequence can be used as a probe to identify and isolate signal sequences and their flanking regions, and these flanking regions can be studied to identify regulatory elements therein.

Described below are TANGO 197 and TANGO 216 polypeptide sequences, any of which can be utilized as part of the fusion polypeptides of the invention.

TANGO 197 and TANGO 216 Polypeptides and Nucleic Acids

The TANGO 197 and TANGO 216 proteins and nucleic acid molecules comprise families of molecules having certain conserved structural and functional features among family members. Examples of conserved structural domains include signal sequence (or signal peptide or secretion signal), transmembrane domains, cytoplasmic domains and extracellular domains. Examples of TANGO 197 and 216 polypeptides are presented herein and can, for example, be utilized as part of the fusion polypeptides of the invention.

TANGO 197

TANGO 197 polypeptides are members of the type A module superfamily, which includes proteins of the extracellular matrix and various proteins with adhesive function, have a von Willebrand factor type A (vWF) domain to which the TANGO 197 proteins of the invention bear similarity. This domain allows for the interaction between various cells and/or extracellular matrix (ECM) components. The domain also contributes to binding to PA. Thus, included within the scope of the invention are TANGO 197 proteins having a von Willebrand factor type A (vWF) domain. As used herein, a vWF domain refers to an amino acid sequence of about 150 to 200, preferably about 160 to 190, 170 to 180, and more preferably about 172 to 175 amino acids in length. A vWF domain of TANGO 197 extends, for example, from about amino acids 44 to 215.

Conserved amino acid motifs, referred to herein as “consensus patterns” or “signature patterns”, can be used to identify TANGO 197 family members having a vWF domain. For example, the following signature pattern can be used to identify TANGO 197 family members: D-x (2)-F-[ILV]-x-D-x-S-x (2, 3)-[ILV]-x (10, 12)-F. The signature patterns or consensus patterns described herein are described according to the following designation: all amino acids are indicated according to their universal single letter designation; “x” designates any amino acid; x(n) designates “n” number of amino acids, e.g., x (2) designates any two amino acids, e.g., x (2, 3) designates any of two to three amino acids; and, amino acids in brackets indicates any one of the amino acids within the brackets, e.g., [ILV] indicates any of one of either I (isoleucine), L (leucine) or V (valine). TANGO 197 has such a signature pattern at about amino acids 44 to 65.

An alignment of TANGO 197 and the vWF consensus sequence is shown in FIG. 2. The vWF consensus sequence is available from the HMMer 2.0 software as Accession Number PF00092. Software for HMM-based profiles is available from http://www.csc.ucsc.edu/research/compbio/sam.html and from http://genome.wustl. edu/eddy/hmmer.html.

In certain embodiments, a TANGO 197 family member has the amino acid sequence of SEQ ID NO:2, and the signal sequence is located at amino acids 1 to 25, 1 to 26, 1 to 27, 1 to 28, or 1 to 29. In such embodiments of the invention, the domains and the mature protein resulting from cleavage of such signal peptides are also included herein. Thus, in another embodiment, a TANGO 197 protein contains a signal sequence of about amino acids 1 to 27 which results in an extracellular domain consisting of amino acids 28 to 301, and a mature TANGO 197 protein corresponding to amino acids 28 to 333 of SEQ ID NO:2. The signal sequence is normally cleaved during processing of the mature protein.

Human TANGO 197

A cDNA encoding a portion of human TANGO 197 was identified by screening a human fetal lung library. An additional screen of an osteoclast library was performed to obtain a clone comprising a full length human TANGO 197. Human TANGO 197 includes a 2272 nucleotide cDNA (FIG. 1; SEQ ID NO:1). It is noted that the nucleotide sequence contains Sal I and Not I adapter sequences on the 5′ and 3′ ends, respectively. The open reading frame of this cDNA (nucleotides 213 to 1211 of SEQ ID NO:11) encodes a 333 amino acid transmembrane protein (SEQ ID NO:2).

The signal peptide prediction program SIGNALP (Nielsen et al. (1997) Protein Engineering 10: 1-6) predicted that human TANGO 197 includes a 27 amino acid signal peptide (amino acids 1 to about amino acid 27 of SEQ ID NO:2) preceding the mature TANGO 197 protein (corresponding to about amino acid 28 to amino acid 333 of SEQ ID NO:2).

Human TANGO 197 includes a vWF domain from about amino acids 44 to 215 of SEQ ID NO:2.

A clone, EpDH213, which encodes human TANGO 197 was deposited as part of EpDHMix1 with the American Type Culture Collection (ATCC®, 10801 University Boulevard, Manassas, Va. 20110-2209) on Nov. 20, 1998 which was assigned Accession Number 98999. This deposit will be maintained under the terms of the Budapest Treaty on the International Recognition of the Deposit of Microorganisms for the Purposes of Patent Procedure. This deposit was made merely as a convenience to those of skill in the art and is not an admission that a deposit is required under 35 U.S.C. §112.

In one embodiment, human TANGO 197 protein is a transmembrane protein that contains an extracellular domain at amino acid residues 28-301 of SEQ ID NO:2, a transmembrane domain at amino acid residues 302 to 319 of SEQ ID NO:2, and a cytoplasmic domain at amino acid residues 320-333 of SEQ ID NO:2. Alternatively, in another embodiment, a human TANGO 197 protein contains an extracellular domain at amino acid residues 320 to 333 of SEQ ID NO:12, a transmembrane domain at amino acid residues 302 to 319 of SEQ ID NO:2, and a cytoplasmic domain at amino acid residues 1 to 301 of SEQ ID NO:2. In yet another embodiment, the human TANGO 197 protein is a secreted protein, the mature form of which comprises amino acid residues 27, 28, 29, or 30 to 333 of SEQ ID NO:2.

Northern analysis of human TANGO 197 mRNA expression revealed expression in a wide variety of tissues such as brain, skeletal muscle, colon, thymus, spleen, kidney, liver, and the small intestine. The highest levels of expression were seen in tissues such as the heart, placenta and lung. There was no expression of the transcript in peripheral blood leukocytes.

Mouse TANGO 197

A mouse homolog of human TANGO 197 was identified. A cDNA encoding part of mouse TANGO 197 was identified by analyzing the sequences of clones present in a mouse testis (Sertoli TM4 cells) cDNA library. This analysis led to the identification of a clone, jtmzb062c08, encoding a partial mouse TANGO 197. The mouse TANGO 197 cDNA of this clone is 4417 nucleotides long (FIG. 3; SEQ ID NO:3). It is noted that the nucleotide sequence contains a Not I adapter sequence on the 3′ end. The open reading frame of this cDNA (nucleotides 3-1145 of SEQ ID NO:3) conceptually translates to a 381 amino acid transmembrane protein (SEQ ID NO:4). Amino acid residues 1 to 3 of this conceptual translation correspond to a linker sequence at the 5′ end, and are not expected to be present in a bona fide full-length mouse TANGO 197 cDNA. Amino acid residues 4 to 135 show a high degree of identity to human TANGO 197. From amino acid residue 136 to 381, the mouse sequence is different from the human sequence, but is predicted to correspond to an alternate form of human TANGO 197. In mouse TANGO 197, amino acid residues 4 to 32 correspond to the C-terminal part of the vWfA domain found in the human TANGO 197 sequence (SEQ ID NO:2). Amino acid residues 33 to 136 do not correspond to a known domain but are still highly similar to those of another protein, human TANGO 216 (SEQ ID NO:6). Amino acid residues 137 to 164 correspond to a predicted transmembrane domain, and amino acid residues 165 to 381 correspond to the cytoplasmic domain. Within this cytoplasmic domain, amino acid residues 165 to 307 are highly similar to the corresponding part of another protein, TANGO 216 suggesting that they form a sub-domain within the cytoplasmic tail. The rest of the cytoplasmic tail from amino acid residues 308 to 381 would form the other half of the subdomain. This second cytoplasmic domain sub-domain is notable for being rich in proline and amino acid residues with uncharged polar side chains (serine, threonine, glutamine and aparagine).

In one embodiment, mouse TANGO 197 protein is a transmembrane protein that contains an extracellular domain at amino acid residues 1 to 136 of SEQ ID NO:4, a transmembrane domain at amino acid residues 137 to 164 of SEQ ID NO:4, and a cytoplasmic domain at amino acid residues 165 to 307 of SEQ ID NO:4.

In another embodiment, mouse TANGO 197 protein is a transmembrane protein that contains an extracellular domain at amino acid residues 161 to 381 of SEQ ID NO:4, a transmembrane domain at amino acid residues 139 to 160 of SEQ ID NO:4, and a cytoplasmic domain at amino acid residues 1 to 138 of SEQ ID NO:4. Alternatively, in another embodiment, a mouse TANGO 197 protein contains an extracellular domain at amino acid residues 1 to 139 of SEQ ID NO:4, a transmembrane domain at amino acid residues 139 to 160 of SEQ ID NO:4, and a cytoplasmic domain at amino acid residues 161 to 381 of SEQ ID NO:4.

Expression of mouse TANGO 197 mRNA was detected by a library array procedure. Briefly, the library array procedure entailed preparing a PCR mixture by adding to the standards reagents (Taq Polymerase, dNTPs, and PCR buffer) a vector primer, a primer internal to the gene of interest, and an aliquot of a library in which expression was to be tested. This procedure was performed with many libraries at a time in a 96 well PCR tray, with 80 or more wells containing libraries and a control well in which the above primers were combined with the clone of interest itself. The control well served as an indicator of the fragment size to be expected in the library wells, in the event the clone of interest was expressed within. Amplification was performed in a PCR machine, employing standard PCR conditions for denaturing, annealing, and elongation, and the resultant mixture was mixed with an appropriate loading dye and run on an ethidium bromide-stained agarose gel. The gel was later viewed with UV light after the DNA loaded within its lanes had time to migrate into the gels. Lanes in which a band corresponding with the control band was visible indicated the libraries in which the clone of interest was expressed.

Results of the library array procedure revealed strong expression in the choroid plexus, 12.5 day whole mouse embryo, LPS-stimulated osteoblast tissue, hyphae stimulated long term bone marrow cells. Weak expression was detected in TM4 (Sertoli cells), from testis, esophagus, LPS-stimulated osteoblast tissue. No expression was detected in differentiated 3T3, 10.5 day mouse fetus, mouse kidney fibrosis model, nephrotoxic serum (NTS), LPS-stimulated heart, LPS-stimulated osteoblasts, lung, mouse insulinoma (Nit-1), normal/hyperplastic islets (pancreas), normal spleen, 11.5 day mouse, LPS-stimulated lung, hypertropic heart, LPS-stimulated kidney, LPS-stimulated lymph node, mc/9 mast cells, 13.5 day mouse, LPS-stimulated anchored heart, normal thymus, Th2-ovarian-Tg, Balb C liver (bile duct ligation d2), normal heart, brain polysome (MPB), LPS-stimulated anchored liver, brain (EAE d10 model), th1-ovarian-Tg, heart, hypothalamus, lone term bone, marrow cells, megakaryocyte, LPS-stimulated spleen, hyphae-stimulated long term bone marrow, lung, angiogenic pancreatic islets, Th2, brain, LPS-stimulated thymus, LPS-stimulated microglial cells, testes (random-primed), tumor pancreatic islets, LPS-stimulated brain, LPS-stimulated alveolar macrophage cell line, mouse lung bleomycin model, pregnant uterus, and hypothalamus nuclei.

Human and mouse TANGO 197 sequences exhibit considerable similarity at the protein, nucleic acid, and open reading frame levels. An alignment (made using the ALIGN software {Myers and Miller (1989) CABIOS, ver. 2.0}; BLOSUM 62 scoring matrix; gap penalties −12/−4), reveals a protein identity of 88.0%. The human and mouse TANGO 197 full length cDNAs are 52.8% identical, as assessed using the same software and parameters as indicated (without the BLOSUM 62 scoring matrix). In the respective ORFs, calculated in the same fashion as the full length cDNAs, human and mouse TANGO 197 are 51.6% identical.

TANGO 216

TANGO 216 proteins include a domain which bears sequence identity to a vWF A domain. Proteins having such a domain are involved in biological processes controlled by specific, often adhesive, molecular interactions. The vWF A domain mediates binding to proteins and sugars. Proteins having vWF A domains may interact through homophilic interactions between vWF A domains. Thus, included are TANGO 216 proteins having a vWF A domain. As used herein, a vWF A domain refers to an amino acid sequence of about 150 to 190, preferably about 155 to 185, 160 to 180, and more preferably about 170 amino acids in length. Conserved amino acid motifs, referred to herein as “consensus patterns” or “signature patterns”, can be used to identify TANGO 216 family members. For example, the following signature pattern can be used to identify TANGO 216 family members: D-x (2)-F-[ILV]-x-D-x-S-x (2, 3)-[ILV]-x (10, 12)-F. TANGO 216 has such a signature pattern at about amino acids 44 to 169 of SEQ ID NO:5.

The vWF A domain consensus sequence is also available from the HMMer version 2.0 software as Accession Number PF00092. Software for HMM-based profiles is available from http://www.csc.ucsc.edu/research/compbio/sam.html and from http://genome.wustl.edu/eddy/hmmer.html. A vWF A domain of TANGO 216 extends, for example, from about amino acids 44 to 213.

In certain embodiments, a TANGO 216 family member has the amino acid sequence, and the signal sequence is located at amino acids 1 to 31, 1 to 32, 1 to 33, 1 to 34 or 1 to 35. In such embodiments of the invention, the domains and the mature protein resulting from cleavage of such signal peptides are also included herein. For example, the cleavage of a signal sequence consisting of amino acids 1 to 33 of SEQ ID NO:6 results in a mature TANGO 216 protein corresponding to amino acids 34 to 488 of SEQ ID NO:6. The signal sequence is normally cleaved during processing of the mature protein.

TANGO 216 proteins also include ones having a transmembrane domain. An example of a transmembrane domain includes from about amino acids 318 to 345 of SEQ ID NO:6.

In one embodiment, a TANGO 216 protein includes a vWF A domain. In another embodiment, a TANGO 216 protein includes a vWF A domain, and a signal sequence. In another embodiment, a TANGO 216 protein includes a vWF A domain, a extracellular domain, and a signal sequence. In another embodiment, a TANGO 216 protein includes a vWF A domain, and an extracellular domain. In another embodiment, a TANGO 216 protein includes a vWF A domain, an extracellular domain, and a transmembrane domain. In another embodiment, a TANGO 216 protein a vWF A domain, an extracellular domain, a transmembrane domain, and a cytoplasmic domain.

Human TANGO 216

The cDNA encoding human TANGO 216 was isolated by screening for cDNAs which encode a potential signal sequence. Briefly, a clone encoding TANGO 216 was isolated through high throughput screening of a prostate stroma cell library. The human TANGO 216 clone includes a 3677 nucleotide cDNA (FIG. 5; SEQ ID NO:5). The open reading frame of this cDNA (nucleotides 307 to 1770 of SEQ ID NO:5), encodes a 488 amino acid transmembrane protein depicted in of SEQ ID NO:6.

In another embodiment, a human TANGO 216 clone comprises a 4350 nucleotide cDNA. The open reading frame of this cDNA comprises nucleotides 353 to 1819, and encodes a the human TANGO 216 transmembrane protein comprising 488 amino acids.

The signal peptide prediction program SIGNALP (Nielsen et al. (1997) Protein Engineering 10: 1-6) predicted that human TANGO 216 includes a 33 amino acid signal peptide (amino acids 1 to about amino acid 33 of SEQ ID NO:6) preceding the mature TANGO 216 protein (corresponding to about amino acid 34 to amino acid 488 of SEQ ID NO:6). The presence of a methionine residue at positions 78, 245, 277, 337, 392, and 369 indicate that there can be alternative forms of human TANGO 216 of 411 amino acids, 244 amino acids, 212 amino acids, 152 amino acids, 97 amino acids, and 120 amino acids of SEQ ID NO:6, respectively.

In one embodiment, human TANGO 216 includes extracellular domains (about amino acids 34 to 79 and 342 to 488), transmembrane (TM) domains (amino acids 80-97 and 318 to 341 of SEQ ID NO:6); and a cytoplasmic domain (amino acids 98 to 317 of SEQ ID NO:6). The cytoplasmic domain is very rich in proline and glutamic acid residues. These residues represent 27% of the residues in the cytoplasmic domain of human TANGO 216.

Alternatively, in another embodiment, a human TANGO 216 protein contains an extracellular domain at amino acid residues 98 to 317, transmembrane (TM) domains (amino acids 80-97 and 318 to 341, and cytoplasmic domains at amino acid residues 1 to 79 and 342-488 of SEQ ID NO:6).

Another embodiment of the invention includes isolated nucleic acid molecules comprising a polynucleotide having a nucleotide sequence encoding the polypeptide having the human TANGO 216 amino acids, but lacking the N-terminal methionine residue. In this embodiment, the nucleotide sequence of human TANGO 216, nucleotides 310-1770, encodes the human TANGO 216 amino acid sequence from amino acids 2-488 of SEQ ID NO:6.

Human TANGO 216 includes a vWF A domain from about amino acids 44 to 213 of SEQ ID NO:6.

Human TANGO 216 protein, including the signal sequence, has a molecular weight of 53.6 kDa prior to post-translational modification. Human TANGO 216 protein has a molecular weight of 50.0 kDa after cleavage of the 33 amino acid signal peptide.

A clone, EpT216, which encodes human TANGO 216 was deposited with the American Type Culture Collection (ATCC®, 10801 University Boulevard, Manassas, Va. 20110-2209) on Mar. 26, 1999, and was assigned Accession Number 207176. This deposit will be maintained under the terms of the Budapest Treaty on the International Recognition of the Deposit of Microorganisms for the Purposes of Patent Procedure. This deposit was made merely as a convenience to those of skill in the art and is not an admission that a deposit is required under 35 U.S.C. §112.

Northern analysis of human TANGO 216 mRNA expression revealed the presence of an approximately 3.8 kb transcript and an approximately 4.3 kb transcript that are expressed in a range of tissues including lung, liver, skeletal muscle, kidney, and pancreas, with highest expression in heart and placenta. The two transcripts likely represent alternative poly A site usage.

The human gene for TANGO 216 was mapped on radiation hybrid panels to the long arm of chromosome 4, in the region q11-13. Flanking markers for this region are GCT14E02 and jktbp-rs2. The JPD (periodontitis, juvenile), and DGI1(dentinogenesis imperfecta) loci also map to this region of the human chromosome. The GRO1 (FRO1 oncogene), ALB (albumin), IL8 (interleukin 8), HTN (histatin), and DCK (deoxycytidine kinase) genes also map to this region of the human chromosome. This region is syntenic to mouse chromosome 5. The rs (recessive spotting) locus also maps to this region of the mouse chromosome. The ste (sulfotransferase), areg (amphiregulin), btc (betacellulin), mc (marcel), alb1 (albumin 1), and afp (alpha fetoprotein) genes also map to this region of the mouse chromosome.

Mouse TANGO 216

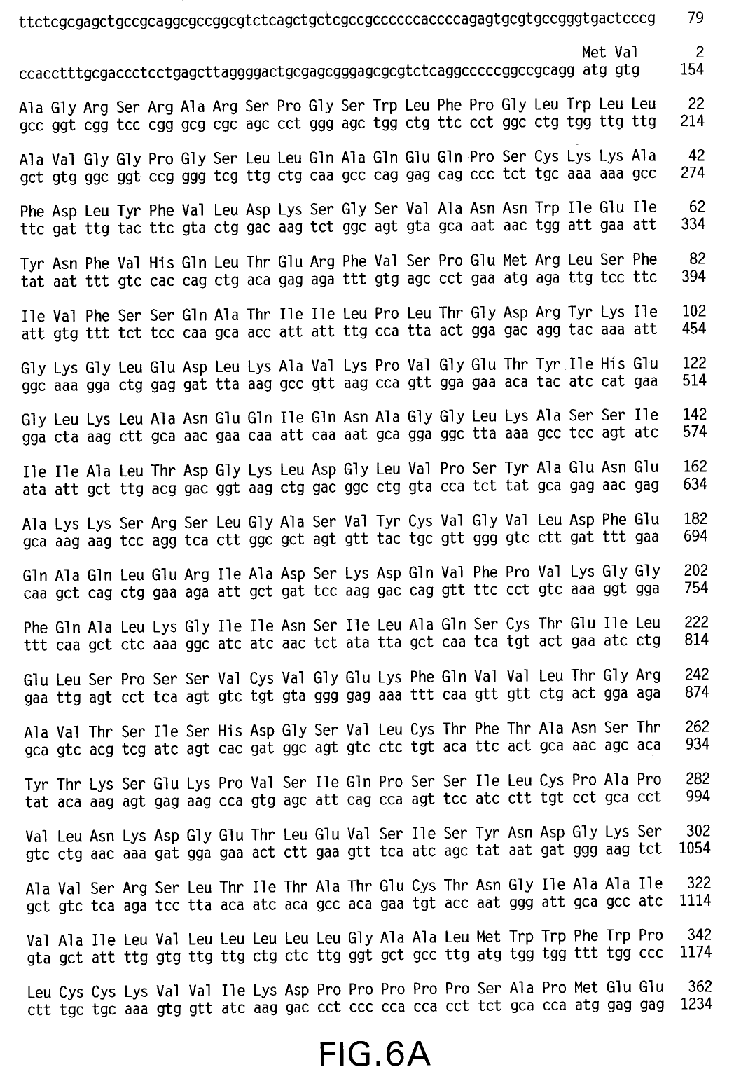

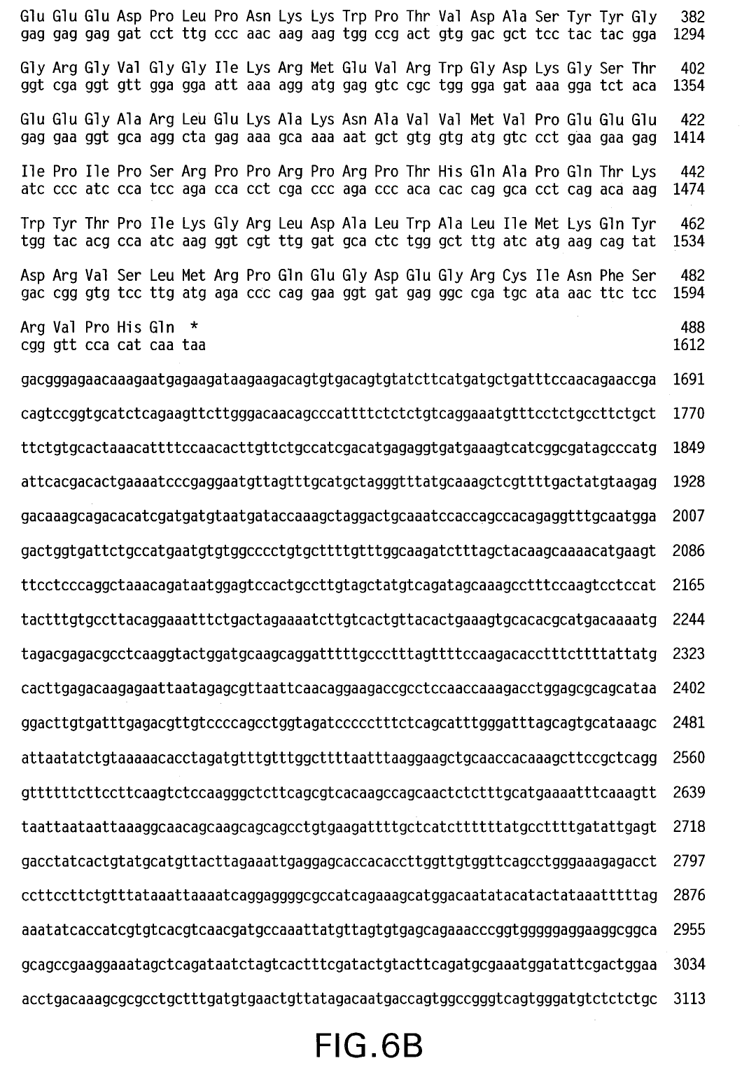

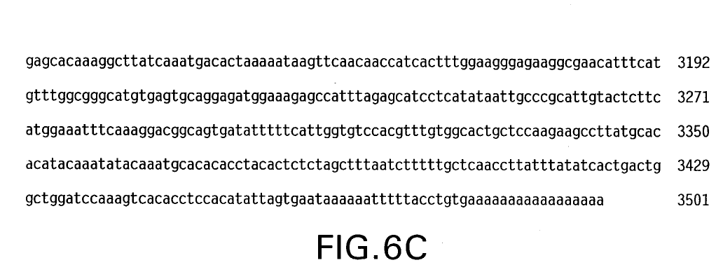

A mouse homolog of human TANGO 216 was identified. A cDNA encoding mouse TANGO 216 was identified by analyzing the sequences of clones present in a mouse bone marrow cDNA library. This analysis led to the identification of a clone, jtmMa005g09, encoding mouse TANGO 216. The mouse TANGO 216 cDNA of this clone is 3501 nucleotides long (FIG. 6; SEQ ID NO:7). The open reading frame of this cDNA (nucleotides 149 to 1609 of SEQ ID NO:7) encodes the 487 amino acid protein depicted in SEQ ID NO:8.

In another embodiment, a mouse TANGO 216 clone comprises a 3647 nucleotide cDNA. The open reading frame of this cDNA comprises nucleotides 32 to 469, and encodes a mouse TANGO 216 transmembrane protein comprising the 146 amino acids.

In one embodiment, mouse TANGO 216 includes extracellular domains (about amino acids 34 to 79 and 342 to 487, transmembrane (TM) domains (amino acids 80-97 and 318 to 341 of SEQ ID NO:8); and a cytoplasmic domain (amino acids 98 to 317 of SEQ ID NO:8). The cytoplasmic domain is very rich in proline and glutamic acid residues. These residues represent 27% of the residues in the cytoplasmic domain of human TANGO 216. Alternatively, in another embodiment, a mouse TANGO 216 protein contains an extracellular domain at amino acid residues 98 to 317, transmembrane (TM) domains (amino acids 80-97 and 318 to 341, and cytoplasmic domains at amino acid residues 1 to 79 and 342-487 of SEQ ID NO:8.

The signal peptide prediction program SIGNALP (Nielsen et al. (1997) Protein Engineering 10: 1-6) predicted that mouse TANGO 216 includes a 33 amino acid signal peptide (amino acids 1 to about amino acid 336 of SEQ ID NO:8) preceding the mature TANGO 216 protein (corresponding to about amino acid 34 to amino acid 487 of SEQ ID NO:8). The presence of a methionine residue at positions 78, 337, 360, 392, 417, 459, and 468 of SEQ ID NO:8 indicate that there can be alternative forms of mouse TANGO 216 of 410 amino acids, 151 amino acids, 128 amino acids, 96 amino acids, 71 amino acids, 29 amino acids, and 20 amino acids of SEQ ID NO:8, respectively.

Mouse TANGO 216 includes a vWF A domain from about amino acids 44 to 213 of SEQ ID NO:8.

Mouse TANGO 216 protein, including the signal sequence, has a molecular weight of 53.2 kDa prior to post-translational modification. Mouse TANGO 216 protein has a molecular weight of 49.8 kDa after cleavage of the 33 amino acid signal peptide.

In situ tissue screening was performed on mouse adult and embryonic tissue to analyze the expression of mouse TANGO 216 mRNA. In the case of adult expression, a low level ubiquitous signal was detected in the spleen and stomach. A weak, ubiquitous signal was detected in the thymus. A ubiquitous signal was detected in the liver, submandibular salivary gland, heart, colon, and in the cortical region of the adrenal gland. A multifocal pattern was detected in the lung and in the decidua of the placenta. A signal was apparent in the villi of the small intestine. No signal was detected in the following tissues: brain, spinal cord, eye, brown fat, white fat, pancreas, skeletal muscle, bladder, kidney, and lung.

In the case of embryonic expression, expression was seen in a number of tissues. At El 3.5, strong signals were detected in the developing spinal column, heart, and tongue. Meckelís cartilage was also apparent. Limb expression is not readily apparent. Low level signal was also seen throughout the gut region including but not restricted to lung, liver, and intestines. Signal is noticeably absent from the developing CNS except for the areas of the brain surrounding the lateral ventricals and mesencephalic vesicle. At E14.5, developing spinal column and sternum, heart, tongue, and Meckelís cartilage continued to have strong signal. Signal from the heart and tongue was ubiqutious. In the brain, the diencephalon had the strongest signal with the areas surrounding the ventricles still being positive. At E15.5, signal was seen in the previously stated regions and was readily seen in the primordium of the basisphenoid bone and primordium of the nasal bone. At E16.5, signal was seen in the previously stated regions, primordium of the basisphenoid bone. At E18.5, the strongest signal was obtained in the developing bone and cartilage areas. Signal from the heart was diminished in strength and now equal to that seen in the rest of the gut region. At P1.5, signal was still strong in the spinal column and nasal septum. Signal was absent from the CNS except for faint signal in the region of the developing cerebellum. Signal is otherwise low and ubiquitous except for heart, small intestine, and stomach which have a slightly higher signal. The highest expressing tissue was the capsule of the kidney which was seen at E14.5 and continues to P1.5.

Human and mouse TANGO 216 sequences exhibit considerable similarity at the protein, nucleic acid, and open reading frame levels. An alignment (made using the ALIGN software (Myers and Miller (1989) CABIOS, ver. 2.0); BLOSUM 62 scoring matrix; gap penalties −12/−4), reveals a protein identity of 84.8%. The human and mouse TANGO 216 full length cDNAs are 84.4% identical, as assessed using the same software and parameters as indicated (without the BLOSUM 62 scoring matrix). In the respective ORFs, calculated in the same fashion as the full length cDNAs, human and mouse TANGO 216 are 84% identical.

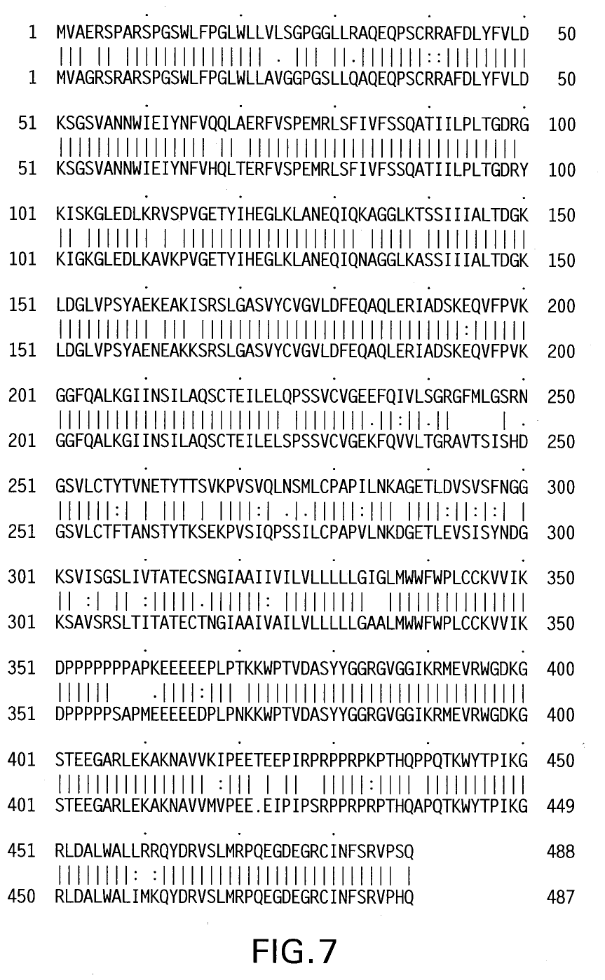

FIG. 7 depicts the alignment of the amino acid sequence of human TANGO 216 and mouse TANGO 216. In this alignment, a (|) between the two sequences indicates an exact match. The depicted alignment of the amino acid sequence of human TANGO 216 (SEQ ID NO:6) and mouse TANGO 216 (SEQ ID NO:8) over 146 amino acids of mouse TANGO 216, indicate a percent identity of approximately 65-68%.

TABLE 1
Summary of Nucleotide Sequence Information of TANGO 197 and
TANGO 216 Nucleic Acids.

| | | | | ATCC |
|---|---|---|---|---|
| | | (OPEN READING FRAME) | | ACCESSION |
| GENE | FIGURE | and CDNA | POLYPEPTIDE | NUMBER |
| h TANGO 197 | | (213-1214 b.p.) | 333 a.a.; | 98999 |
| | | 2272 b.p.; SEQ ID NO:2 | | |
| | | SEQ ID NO:1 | | |
| m TANGO 197 | | (3-1145 b.p.) | 381 a.a.; | |
| | | 4417 b.p.; SEQ ID NO:4 | | |
| | | SEQ ID NO:3 | | |
| h TANGO 216 | | (307-1770 b.p.) | 488 a.a.; | 207176 |
| | | 3677 b.p.; SEQ ID NO:6 | | |
| | | | SEQ ID NO:5 | |
| m TANGO 216 | | (149-1609 b.p.) | 487 a.a.; | |
| | | 3501 b.p.; | SEQ ID NO:8 | |
| | | SEQ ID NO:7 | | |

The invention can utilize fragments of any of the polypeptides described herein wherein the fragment retains a biological or structural function by which the full-length polypeptide is characterized (e.g., an activity or a binding capacity). The invention furthermore includes fragments of any of the polypeptides described herein wherein the fragment has an amino acid sequence sufficiently (e.g., 50%, 60%, 70%, 80%, 85%, 90%, 95%, 98%, or 99% or greater) identical to the amino acid sequence of the corresponding full-length polypeptide that it retains a biological or structural function by which the full-length polypeptide is characterized (e.g., an activity or a binding capacity).

Also within the invention are fusion polypeptides having an amino acid sequence that is at least about 50%, preferably 60%, 75%, 90%, 95%, or 98% identical to the amino acid sequence of any of SEQ ID NOs:2, 4, 6, and 8.

Also within the invention are fusion polypeptides comprising naturally occurring allelic variants of a polypeptide that includes the amino acid sequence of any of SEQ ID NOs:2, 4, 6, and 8, wherein the polypeptide is encoded by a nucleic acid molecule which hybridizes under stringent conditions to a nucleic acid molecule having the nucleotide sequence of any of SEQ ID NOs:1, 3, 5, and 7, and the nucleotide sequence of any of the clones deposited as ATCC® Accession numbers 98999 and 207176, or a complement thereof.

In another embodiment, a fusion polypeptide of the invention has an amino acid sequence sufficiently identical to an identified domain of a TANGO 197 or 216 polypeptide. As used herein, the term “sufficiently identical” refers to a first amino acid or nucleotide sequence which contains a sufficient or minimum number of identical or equivalent (e.g., with a similar side chain) amino acid residues or nucleotides to a second amino acid or nucleotide sequence such that the first and second amino acid or nucleotide sequences have or encode a common structural domain and/or common functional activity. For example, amino acid or nucleotide sequences which contain or encode a common structural domain having about 60% identity, preferably about 65% identity, more preferably about 75%, 85%, 95%, 98% or more identity are defined herein as sufficiently identical.

The present invention also pertains to fusion polypeptides that act as variants of TNAGO 197 or TANGO 216. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a protein of the invention which function as either agonists (mimetics) or as antagonists can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the invention from a degenerate oligonucleotide sequence. Methods for synthesizing degenerate oligonucleotides are known in the art (see, e.g., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

Uses for the TANGO 197 and TANGO 216 Fusion Polypeptides of the Invention

In one aspect, the present invention provides a method for preventing a symptom of anthrax in a subject thought to be at risk for exposure to Bacillus anthracis, comprising: administering to the subject a pharmaceutically effective amount of a fusion polypeptide so that, if the subject is exposed to Bacillus anthracis, a symptom of said exposure is prevented, wherein said fusion polypeptide comprises a von Willebrand factor A-like domain (vWF) amino acid sequence and an amino acid sequence heterologous to said vWF. Pharmaceutical compositions comprising a fusion polyeptide of the invention can be as described below.

For example, in such an embodiment, administration of the fusion polypeptide can take place prior to a subject's exposure to an environment or situation that places the subject at risk for exposure to Bacillus anthracis. In such an instance, a pharmaceutically effective amount of the fusion polypeptide represents an amount sufficient to yield a fusion polypeptide concentration competent to prevent a symptom of anthrax in the subject if said subject is, indeed, exposed to the bacterium. While particular administration dosages will depend upon the particular circumstances, typical administration is administration of about 1 to 10, 1 to 25, 1 to 50, or 1 to 100 mg/kg body weight, administered once weekly. Typical administration route is subcutaneous or intramuscular.

In another aspect, the present invention provides a method for preventing a symptom of anthrax in a subject suspected of having been exposed to Bacillus anthracis, comprising: administering to the subject a pharmaceutically effective amount of a fusion polypeptide so that, if the subject has been exposed to Bacillus anthracis, a symptom of said exposure is prevented, wherein said fusion polypeptide comprises a von Willebrand factor A-like domain (vWF) amino acid sequence and an amino acid sequence heterologous to said vWF.

In such an embodiment, for example, administration of the fusion polypeptide can take upon a subject's suspected or putative exposure to to Bacillus anthracis. As above, in such an instance, a pharmaceutically effective amount of the fusion polypeptide represents an amount sufficient to yield a fusion polypeptide concentration competent to prevent a symptom of anthrax in the subject if said subject is, indeed, exposed to the bacterium. While particular administration dosages will depend upon the particular circumstances, typical administration is administration of about 1 to 10, 1 to 25, 1 to 50, or 1 to 100 mg/kg body weight, administered once weekly. Typical administration route is subcutaneous or intramuscular, with intramuscular being preferred.

In yet another aspect, the present invention provides a method for ameliorating a symptom of anthrax in a subject in need of said amelioration, comprising: administering to the subject a pharmaceutically effective amount of a fusion polypeptide so that a symptom of anthrax is ameliorated, wherein said fusion polypeptide comprises a von Willebrand factor A-like domain (vWF) amino acid sequence and an amino acid sequence heterologous to said vWF.

In such an embodiment, a pharmaceutically effective amount of the fusion polypeptide represents the amount necessary to produce a fusion polypeptide concentration sufficient to ameliorate a symptom of anthrax. Symptoms of anthrax, both cutaneous and inhalation, are well known. For example, symptoms include high fever, radiographic evidence of pneumonia, petechia, and coagulopathy. While particular administration dosages will depend upon the particular circumstances, typical administration is administration of about 1 to 10, 1 to 25, 1 to 50, or 1 to 100 mg/kg body weight, administered once daily, weekly, or monthly. Typical administration route is subcutaneous, intramuscular, or intravenous, with intramuscular being preferred, and intravenous most preferred.

In still another embodiment, the fusion polypeptides of the invention, can be utilized as markers for identification of cancer cells. This is due to high level of homology between the TANGO 197 and TANGO 216 sequences comprising the fusion polypeptides of the invention and TEM8 a cell surface tumor endothelial cancer marker (Carson-Walter, E. B. et al. Cancer Res 61, 6649-6655 (2001)). For example, the fusion polypeptides of the invention can be utilized to generate diagnostic or therapeutic antibodies against a cancer, e.g., tumor, cell and/or against tumor-related vasculature.

In addition to the above, the TANGO 197 and TANGO 216 fusion polypeptides can be used as part of additional methods, as described below:

As TANGO 197 exhibits expression in the lung, TANGO 197 polypeptides, fusion polypeptides comprising TANGO 197, and/or nucleic acids, or modulators thereof, can be used to treat, diagnosis, prognose or detect pulmonary (lung) disorders, such as atelectasis, pulmonary congestion or edema, chronic obstructive airway disease (e.g., emphysema, chronic bronchitis, bronchial asthma, and bronchiectasis), diffuse interstitial diseases (e.g., sarcoidosis, pneumoconiosis, hypersensitivity pneumonitis, Goodpasture's syndrome, idiopathic pulmonary hemosiderosis, pulmonary alveolar proteinosis, desquamative interstitial pneumonitis, chronic interstitial pneumonia, fibrosing alveolitis, hamman-rich syndrome, pulmonary eosinophilia, diffuse interstitial fibrosis, Wegener's granulomatosis, lymphomatoid granulomatosis, and lipid pneumonia), or tumors (e.g., bronchogenic carcinoma, bronchiolovlveolar carcinoma, bronchial carcinoid, hamartoma, and mesenchymal tumors).

Morever, as a species isoform of TANGO 197 was also isolated from a testis library, therefore TANGO 197 polypeptides, fusion polypeptides comprising TANGO 197, and/or nucleic acids, or modulators thereof, can be used to treat, diagnose or detect testicular disorders, such as unilateral testicular enlargment (e.g., nontuberculous, granulomatous orchitis), inflammatory diseases resulting in testicular dysfunction (e.g., gonorrhea and mumps), and tumors (e.g., germ cell tumors, interstitial cell tumors, androblastoma, testicular lymphoma and adenomatoid tumors).

Furthermore, as TANGO 197 is expressed in the testis, the TANGO 197 polypeptides, fusion polypeptides comprising TANGO 197 amino acid sequences, and/or nucleic acids and/or modulators thereof can be used to modulate, detect or diagnose for example and without limitation, Klinefelter syndrome (both the classic and mosaic forms), XX male syndrome, variococele, germinal cell aplasia (the Sertoli cell-only syndrome), idiopathic azoospermia or severe oligospermia, crpytochidism, and immotile cilia syndrome, or testicular cancer (primary germ cell tumors of the testis). In another example, TANGO 197 polypeptides, fusion polypeptides comprising TANGO 197 amino acid sequences, and/or nucleic acids, or modulators thereof, can be used to treat, diagnose or detect testicular disorders, such as unilateral testicular enlargment (e.g., nontuberculous, granulomatous orchitis), inflammatory diseases resulting in testicular dysfunction (e.g., gonorrhea and mumps), and tumors (e.g., germ cell tumors, interstitial cell tumors, androblastoma, testicular lymphoma and adenomatoid tumors).

As discussed above, the vWF domain of TANGO 197 is involved in cellular adhesion and interaction with extracellular matrix (ECM) components. Proteins of the type A module superfamily which incorporate a vWF domain participate in multiple ECM and cell/ECM interactions. For example, proteins having a vWF domain have been found to play a role in cellular adhesion, migration, homing, pattern formation and/or signal transduction after interaction with several different ligands (Colombatti et al. (1993) Matrix 13:297-306).

TANGO 197 polypeptides, fusion polypeptides comprising TANGO 197 amino acid sequences, and/or nucleic acids and/or modulators thereof can also be used to modulate, diagnose or detect cell adhesion in proliferative disorders, such as cancer. Examples of types of cancers include benign tumors, neoplasms or tumors (such as carcinomas, sarcomas, adenomas or myeloid lymphoma tumors, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leimyosarcoma, rhabdotheliosarcoma, colon sarcoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hematoma, bile duct carcinoma, melanoma, choriocarcinoma, semicoma, embryonal carcinoma, Wilms' tumor, cervical cancer, testicular tumor, lung carcinoma, small cell carcinoma, bladder carcinoma, epithelial carcinoma, glioma, astrocytoma, medulloblastoma, craniopharyngioma, ependynoma, pinealoma, hemangioblastoma, retinoblastoma), leukemias, (e.g. acute lymphocytic leukemia), acute myelocytic leukemia (myelolastic, promyelocytic, myelomonocytic, monocytic and erythroleukemia), chronic leukemias (chronic myelocytic (granulocytic) leukemia and chronic lymphocytic leukemia), or polycythemia vera, or lymphomas (Hodgkin's disease and non-Hodgkin's diseases), multiple myelomas and Waldenström's macroglobulinemia.

TANGO 216 proteins include a vWF A domain. Accordingly, TANGO 216 proteins likely function in a similar manner as other proteins which include a vWF A domain, including von Willebrand factor, a large multimeric protein found in platelets, endothelial cells, and plasma. Thus, TANGO 216 modulators can be used to treat any von Willebrand factor-associated disorders and modulate normal von Willebrand factor functions, including processes involved, either directly or indirectly, with anthrax and symptoms associated with exposure to anthrax.

As discussed above, the vWF domain of TANGO 216 is involved in cellular adhesion and interaction with extracellular matrix (ECM) components. Proteins of the type A module superfamily which incorporate a vWF domain participate in multiple ECM and cell/ECM interactions. For example, proteins having a vWF domain have been found to play a role in cellular adhesion, migration, homing, pattern formation and/or signal transduction after interaction with several different ligands (Colombatti et al. (1993) Matrix 13:297-306).

Similarly, the TANGO 216 proteins likely play a role in various extracellular matrix interactions, e.g., matrix binding, and/or cellular adhesion. Thus, a TANGO 216 activity is at least one or more of the following activities: 1) regulation of extracellular matrix structuring; 2) modulation of cellular adhesion, either in vitro or in vivo; 3) regulation of cell trafficking and/or migration. Accordingly, the TANGO 216 proteins, fusion polypeptides, and/or nucleic acid molecules and/or modulators can be used to modulate or detect cellular interactions such as cell-cell and/or cell-matrix interactions and thus, to treat disorders associated with abnormal cellular interactions.

TANGO 216 polypeptides, fusion polypeptides, and/or nucleic acids and/or modulators thereof can also be used to modulate, detect or diagnose cell adhesion in proliferative disorders, such as cancer. Examples of types of cancers include benign tumors, neoplasms or tumors (such as carcinomas, sarcomas, adenomas or myeloid lymphoma tumors, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leimyosarcoma, rhabdotheliosarcoma, colon sarcoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hematoma, bile duct carcinoma, melanoma, choriocarcinoma, semicoma, embryonal carcinoma, Wilms' tumor, cervical cancer, testicular tumor, lung carcinoma, small cell carcinoma, bladder carcinoma, epithelial carcinoma, glioma, astrocytoma, medulloblastoma, craniopharyngioma, ependynoma, pinealoma, hemangioblastoma, retinoblastoma), leukemias, (e.g. acute lymphocytic leukemia), acute myelocytic leukemia (myelolastic, promyelocytic, myelomonocytic, monocytic and erythroleukemia), chronic leukemias (chronic myelocytic (granulocytic) leukemia and chronic lymphocytic leukemia), or polycythemia vera, or lymphomas (Hodgkin's disease and non-Hodgkin's diseases), multiple myelomas and Waldenström's macroglobulinemia.

As TANGO 216 was originally isolated from a bone marrow library, TANGO 216 nucleic acids, proteins, fusion polypeptides, and modulators thereof can be used to modulate, detect and/or diagnose the proliferation, differentiation, and/or function of cells that appear in the bone marrow, e.g., stem cells (e.g., hematopoietic stem cells), and blood cells, e.g., erythrocytes, platelets, and leukocytes. Thus TANGO 216 nucleic acids, proteins, fusion polypeptides, and modulators thereof can be used to treat, detect, and diagnose bone marrow, blood, and hematopoietic associated diseases and disorders, e.g., acute myeloid leukemia, hemophilia, leukemia, anemia (e.g., sickle cell anemia), and thalassemia.

As TANGO 216 exhibits expression in the embryonic lung, TANGO 216 polypeptides, fusion polypeptides, nucleic acids, or modulators thereof, can be used to treat, detect, and diagnose pulmonary (lung) disorders, such as atelectasis, pulmonary congestion or edema, chronic obstructive airway disease (e.g., emphysema, chronic bronchitis, bronchial asthma, and bronchiectasis), diffuse interstitial diseases (e.g., sarcoidosis, pneumoconiosis, hypersensitivity pneumonitis, Goodpasture's syndrome, idiopathic pulmonary hemosiderosis, pulmonary alveolar proteinosis, desquamative interstitial pneumonitis, chronic interstitial pneumonia, fibrosing alveolitis, hamman-rich syndrome, pulmonary eosinophilia, diffuse interstitial fibrosis, Wegener's granulomatosis, lymphomatoid granulomatosis, and lipid pneumonia), or tumors (e.g., bronchogenic carcinoma, bronchiolovlveolar carcinoma, bronchial carcinoid, hamartoma, and mesenchymal tumors).

As TANGO 216 exhibits expression in the small intestine, TANGO 216 polypeptides, fusion polypeptides, nucleic acids, or modulators thereof, can be used to treat, detect, or diagnose intestinal disorders, such as ischemic bowel disease, infective enterocolitis, Crohn's disease, benign tumors, malignant tumors (e.g., argentaffinomas, lymphomas, adenocarcinomas, and sarcomas), malabsorption syndromes (e.g., celiac disease, tropical sprue, Whipple's disease, and abetalipoproteinemia), obstructive lesions, hernias, intestinal adhesions, intussusception, or volvulus.

As TANGO 216 exhibits expression in the spleen, TANGO 216 nucleic acids, proteins, fusion polypeptides, and modulators thereof can be used to modulate the proliferation, differentiation, and/or function of cells that form the spleen, e.g., cells of the splenic connective tissue, e.g., splenic smooth muscle cells and/or endothelial cells of the splenic blood vessels. TANGO 216 nucleic acids, proteins, and modulators thereof can also be used to modulate, detect or diagnose the proliferation, differentiation, and/or function of cells that are processed, e.g., regenerated or phagocytized within the spleen, e.g., erythrocytes and/or B and T lymphocytes and macrophages. Thus, TANGO 216 nucleic acids, proteins, fusion polypeptides, and modulators thereof can be used to treat, detect or diagnose spleen, e.g., the fetal spleen, associated diseases and disorders. Examples of splenic diseases and disorders include e.g., splenic lymphoma and/or splenomegaly, and/or phagocytotic disorders, e.g., those inhibiting macrophage engulfment of bacteria and viruses in the bloodstream.

As TANGO 216 is expressed in the kidney, the TANGO 216 polypeptides, fusion polypeptides, nucleic acids and/or modulators thereof can be used to modulate, detect or diagnose the function, morphology, proliferation and/or differentiation of cells in the tissues in which it is expressed. Such molecules can also be used to treat, detect or diagnose disorders associated with abnormal or aberrant metabolism or function of cells in the tissues in which it is expressed. Such can be used to treat or modulate renal (kidney) disorders, such as glomerular diseases (e.g., acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, neoplasia, sickle cell disease, and chronic inflammatory diseases), tubular diseases (e.g., acute tubular necrosis and acute renal failure, polycystic renal disease, medullary sponge kidney, medullary cystic disease, nephrogenic diabetes, and renal tubular acidosis), tubulointerstitial diseases (e.g., pyelonephritis, drug and toxin induced tubulointerstitial nephritis, hypercalcemic nephropathy, and hypokalemic nephropathy) acute and rapidly progressive renal failure, chronic renal failure, nephrolithiasis, vascular diseases (e.g., hypertension and nephrosclerosis, microangiopathic hemolytic anemia, atheroembolic renal disease, diffuse cortical necrosis, and renal infarcts), or tumors (e.g., renal cell carcinoma and nephroblastoma).

As TANGO 216 exhibits expression in the heart, TANGO 216 polypeptides, fusion polypeptides, nucleic acids, or modulators thereof, can be used to treat cardiovascular disorders as described herein.

As TANGO 216 exhibits expression in bone structures, TANGO 216 nucleic acids, fusion polypeptides, proteins, and modulators thereof can be used to modulate the proliferation, differentiation, and/or function of bone and cartilage cells, e.g., chondrocytes and osteoblasts, and to treat bone and/or cartilage associated diseases or disorders. Examples of bone and/or cartilage diseases and disorders include bone and/or cartilage injury due to for example, trauma (e.g., bone breakage, cartilage tearing), degeneration (e.g., osteoporosis), degeneration of joints, e.g., arthritis, e.g., osteoarthritis, and bone wearing.

The extracellular region of TANGO 216 has significant similarity to TANGO 197, a secreted protein. TANGO 197 has a vWF A domain and may interact with TANGO 216.

TANGO 216 likely plays a role in the regulation of binding of cells in circulation to the endothelial substrate. Thus, TANGO 216 may regulate proper flow of cells in the heart, vasculature, and placenta. Accordingly, the TANGO 216 proteins, fusion polypeptides, nucleic acids and/or modulators of the invention are useful modulators of interactions between cells in circulation and endothelial substrate which can be used to treat, diagnose or detect disorders of such interactions.

In terms of the homology between TANGO 197 and TANGO 216, human TANGO 197 and Human TANGO 216 sequences exhibit substantial similarity at the protein, nucleic acid, and open reading frame levels. An alignment (made using the ALIGN software {Myers and Miller (1989) CABIOS, ver. 2.0}; BLOSUM 62 scoring matrix; gap penalties −12/−2), reveals a protein identity of 48.8% (FIG. 8). The human TANGO 197 and human TANGO 216 full length cDNAs are 44.8% identical, as assessed using the same software and parameters as indicated (without the BLOSUM 62 scoring matrix). In the respective ORFs, calculated in the same fashion as the full length cDNAs, human TANGO 197 and human TANGO 216 are 43.1% identical.

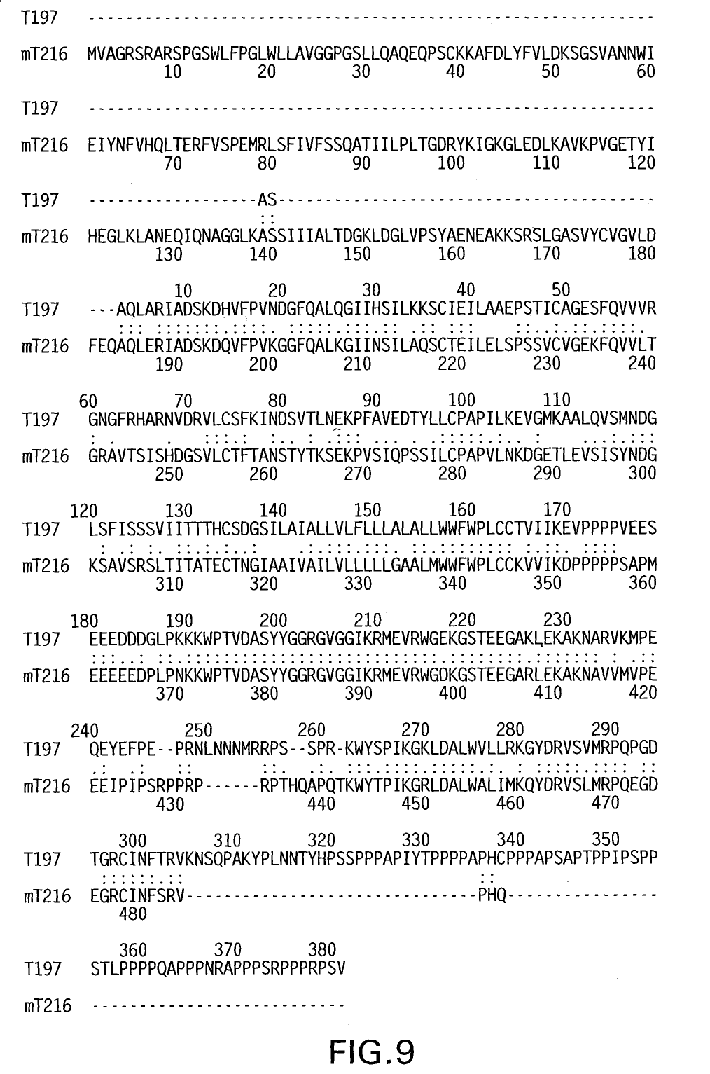

Mouse TANGO 197 and mouse TANGO 216 sequences exhibit substantial similarity at the protein, nucleic acid, and open reading frame levels. An alignment (made using the ALIGN software {Myers and Miller (1989) CABIOS, ver. 2.0}; BLOSUM 62 scoring matrix; gap penalties −12/−2), reveals a protein identity of 48.8% (FIG. 9). The mouse TANGO 197 and mouse TANGO 216 full length cDNAs are 44.8% identical, as assessed using the same software and parameters as indicated (without the BLOSUM 62 scoring matrix). In the respective ORFs, calculated in the same fashion as the full length cDNAs, mouse TANGO 197 and mouse TANGO 216 are 43.1% identical. Based upon the homology exhibited between TANGO 197 and TANGO 216, particularly in the region of the vWFA domain, it is predicted that fusion polypeptides comprising a von Willebrand factor A-like domain (vWF) amino acid sequence of TANGO 216 and an amino acid sequence heterologous to said vWF can be utilized in methods of the present invention.

Production of the Fusion Polypeptides of the Invention

In one embodiment, fusion polypeptides of the invention are produced by recombinant DNA techniques. Alternative to recombinant expression, a fusion polypeptide of the invention can be synthesized chemically using standard peptide synthesis techniques.

Expression vectors can routinely be designed for expression of a fusion polypeptide of the invention in prokaryotic (e.g., E. coli) or eukaryotic cells (e.g., insect cells (using baculovirus expression vectors), yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerivisae include pYepSec1 (Baldari et al. (1987) EMBO J. 6:229-234), pMFa (Kurjan and Herskowitz, (1982) Cell 30:933-943), pJRY88 (Schultz et al. (1987) Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the alpha-fetoprotein promoter (Campes and Tilghman (1989) Genes Dev. 3:537-546).

A host cell can be any prokaryotic (e.g., E. coli) or eukaryotic cell (e.g., insect cells, yeast or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

Pharmaceutical Compositions

Fusion polypeptides of the invention can be incorporated into pharmaceutical compositions suitable for administration. Such compositions typically comprise the fusion protein, and a pharmaceutically acceptable carrier, excipient or diluent. As used herein the language “pharmaceutically acceptable carrier, excipient or diluent” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions. Such methods comprise formulating a pharmaceutically acceptable carrier with an agent which modulates expression or activity of a polypeptide of the invention. Such compositions can further include additional active agents. Thus, the invention further includes methods for preparing a pharmaceutical composition by formulating a pharmaceutically acceptable carrier with an agent which modulates expression or activity of a polypeptide of the invention and one or more additional active compounds.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

As defined herein, a therapeutically effective amount of protein or polypeptide (i.e., an effective dosage) ranges from about 0.001 to 30, 50 or 100 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a fusion protein, can include a single treatment or, preferably, can include a series of treatments. It will also be appreciated that the effective dosage of polypeptide used for treatment may increase or decrease over the course of a particular treatment. Changes in dosage may result and become apparent from the results of diagnostic assays as described herein.

The pharmaceutical compositions can be included in a container, pack, or dispenser together with instructions for administration.

### EXAMPLE

### Construction of T197 Fusion Proteins

This example describes the construction of nucleic acids that encode T197 fusion proteins and nucleic acids that encode the fusion proteins. The T197 fusion proteins comprise T197 extracellular domains, including the von Willebrand factor domain A-like region and sequences fused to one or more additional heterologous protein sequences. The T197 fusion proteins described herein can be utilized for a number of uses including use as antioxins as part of anthrax prophylactic and/or therapeutic methods.

Nine T197 fusion proteins, pLKTOK125, pLKTOK126, pLKTOK127 and pLKTOK129, pO610, pO611, pO613, pO614 and pO615, were constructed as described herein.

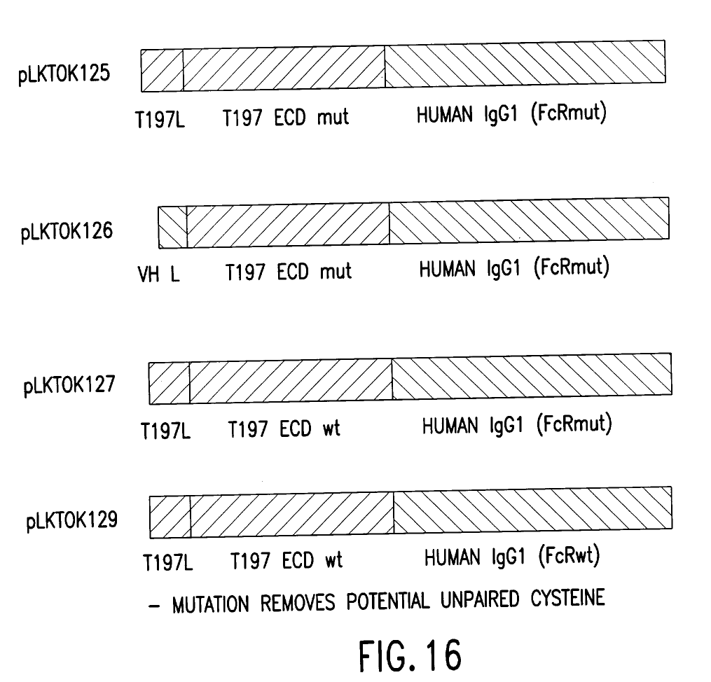

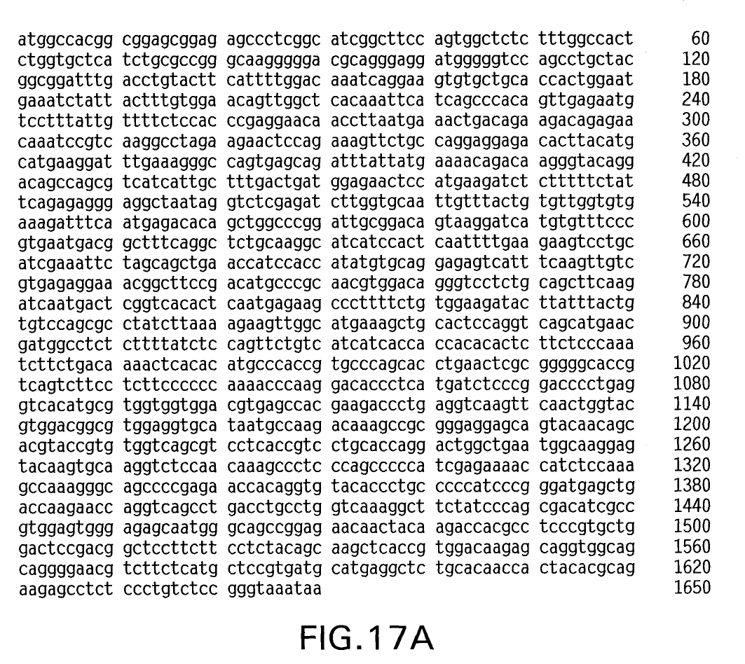

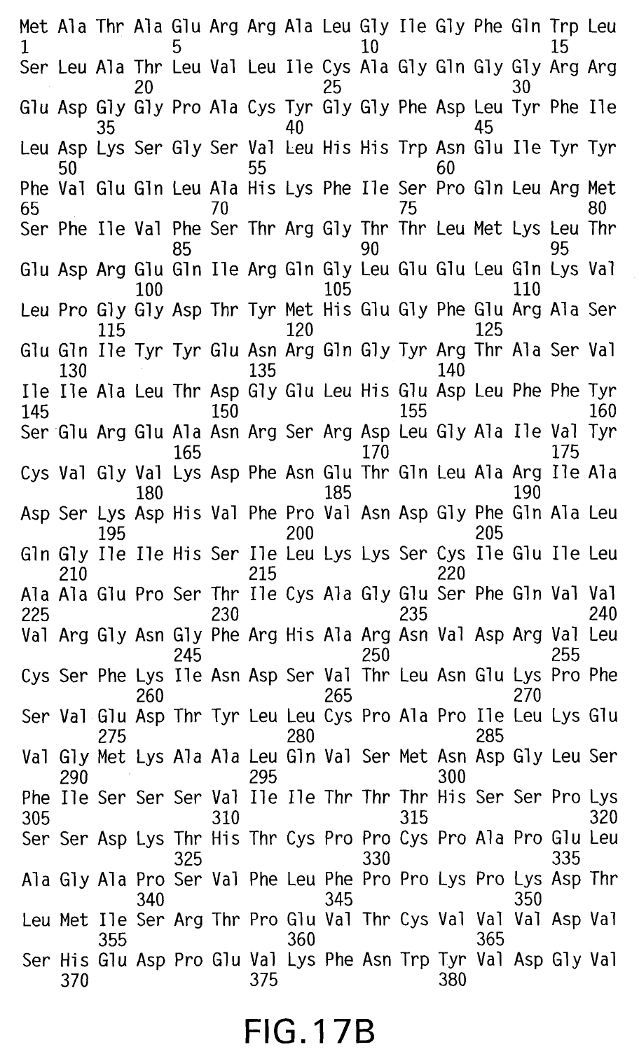

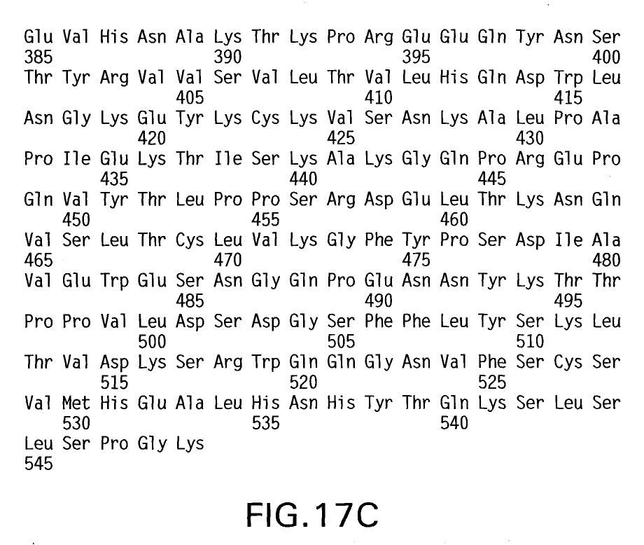

pLKTOK125: The amino terminus of the mature form of the pLKTOK125 protein comprises the extracellular region of human TANGO 197, minus the sequence of the two amino acids (DG) closest to the transmembrane region. This was done because the sequence can lower fusion protein stability when utilized with an Ig heterologous sequence. As such, it is noted that when using a TANGO 197 sequence in conjunction with an Ig heterologous sequence that it is preferred that these two amino acid residues be removed. In addition, this sequence contains a serine amino acid residue at position 317 (bold in FIG. 17) in place of the cysteine that is present in the wild type sequence. The carboxy terminus of the pLKTOK125 protein comprises the Fc sequence of human IgG1 with mutations at positions 235 (a leucine to alanine mutation relative to wild type) and 237 (a glycine to alanine mutation relative to wild type). These Fc mutations inhibit binding of the constant region to human Fc receptors and also inhibit the initiation of ADCC reaction. See U.S. Pat. No. 5,985,279 and PCT Publication No. WO 98/06428. The immature form of the pLKTOK125 protein further comprises a human T197 signal peptide sequence. A diagrammatic depiction of the pLKTOK125 protein is shown in FIG. 16. The amino acid sequence of the pLKTOK125 protein is shown in FIG. 17 (SEQ ID NO:10); the serine residue presented in bold represents the residue that has been mutated from cysteine, and the LAGA residues presented in bold represent the Fc sequence that has been mutated as described above. The nucleotide sequence encoding the pLKTOK125 protein is shown in FIG. 17 (SEQ ID NO:9).

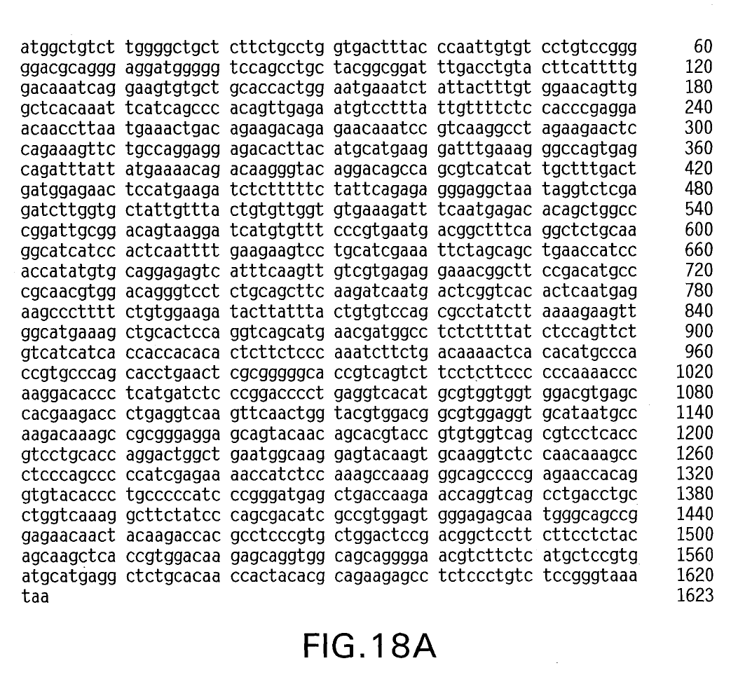

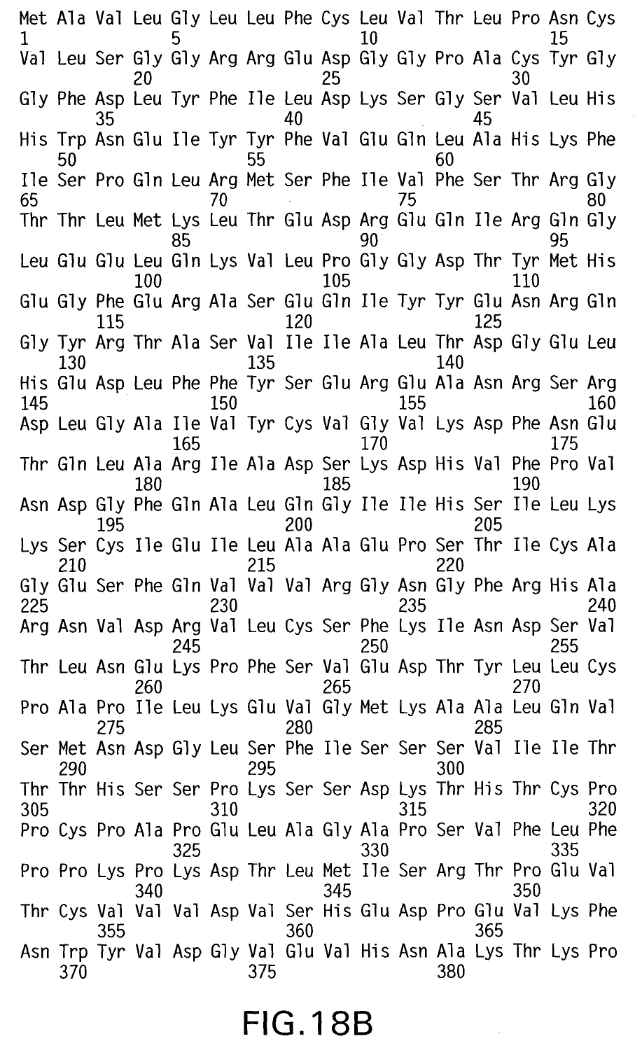

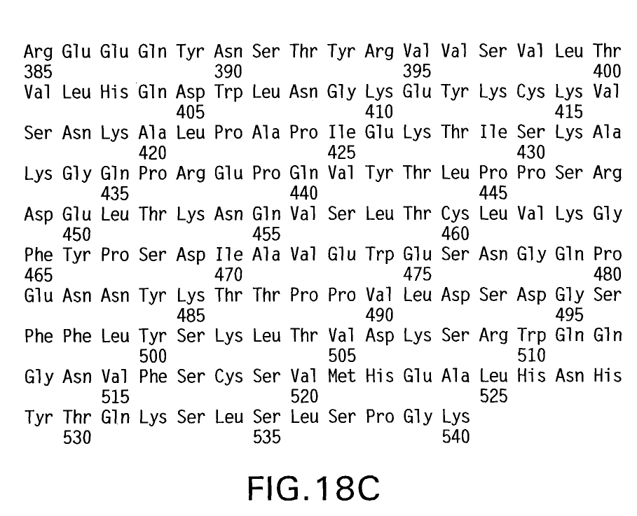

pLKTOK126: The amino terminus of the mature form of the pLKTOK125 protein comprises the extracellular region of human T197, minus the sequence of the two amino acids closest to the transmembrane region for reasons set forth above. In addition, this sequence contains a serine amino acid residue at position 317 in place of the cysteine that is present in the wild type sequence. The carboxy terminus of the pLKTOK126 protein comprises the Fc sequence of human IgG1 with mutations at positions 235 (a leucine to alanine mutation relative to wild type) and 237 (a glycine to alanine mutation relative to wild type). The immature form of the pLKTOK126 protein further comprises a human Ig signal peptide sequence ad described in copending patent application identified by internal reference number MP12001-244P1(M), filed Oct. 19, 2001, the entire contents of which are incorporated herein by reference in its entirety. A diagrammatic depiction of the pLKTOK126 protein is shown in FIG. 16. The amino acid sequence of the pLKTOK126 protein is shown in FIG. 18 (SEQ ID NO:12); the serine residue presented in bold represents the residue that has been mutated from cysteine, and the LAGA residues presented in bold represent the Fc sequence that has been mutated as described above. The nucleotide sequence encoding the pLKTOK126 protein is shown in FIG. 18 (SEQ ID NO:11).

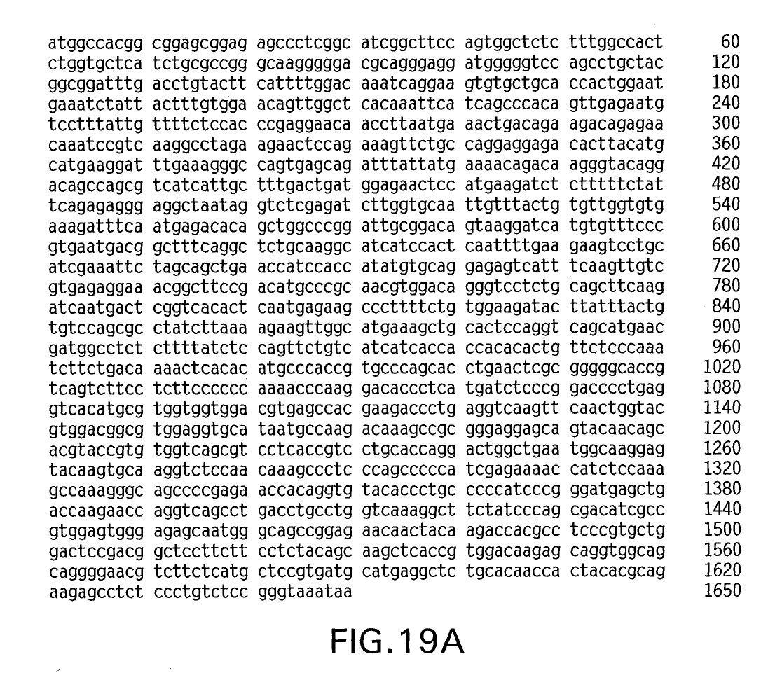

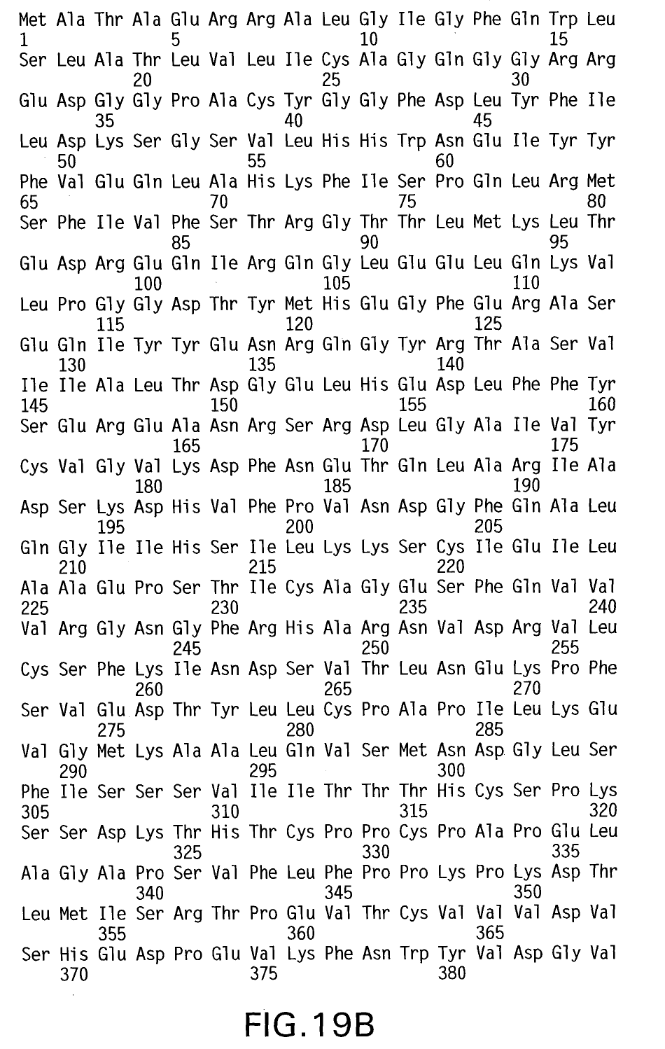

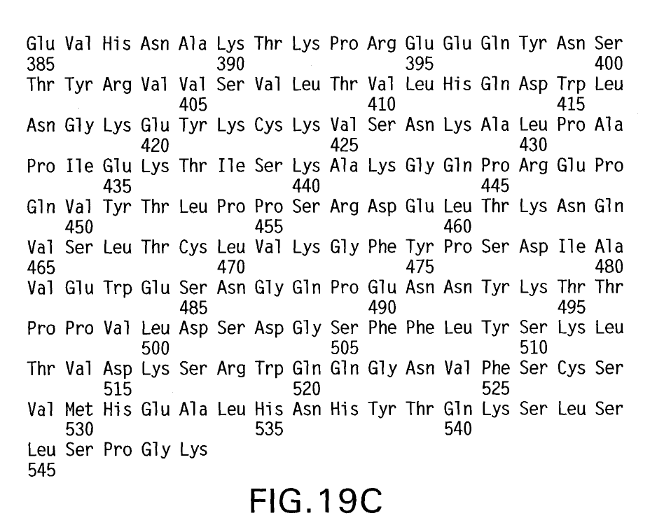

pLKTOK127: The amino terminus of the mature form of the pLKTOK127 protein comprises the extracellular region of human T197, minus the sequence of the two amino acids closest to the transmembrane region for the reasons presented above. The carboxy terminus of the pLKTOK127 protein comprises the Fc sequence of human IgG1 with mutations at positions 235 (a leucine to alanine mutation relative to wild type) and 237 (a glycine to alanine mutation relative to wild type). The immature form of the pLKTOK127 protein contains a human T197 signal peptide sequence. A diagrammatic depiction of the pLKTOK127 protein is shown in FIG. 16. The amino acid sequence of the pLKTOK127 protein is shown in FIG. 19 (SEQ ID NO:14); the cysteine residue presented in bold represents the wild type residue that has been mutated to serine in the pLKTOK125 and pLKTOK126 proteins, and the LAGA residues presented in bold represent the Fc sequence that has been mutated as described above. The nucleotide sequence encoding the pLKTOK127 protein is shown in FIG. 19 (SEQ ID NO:13).

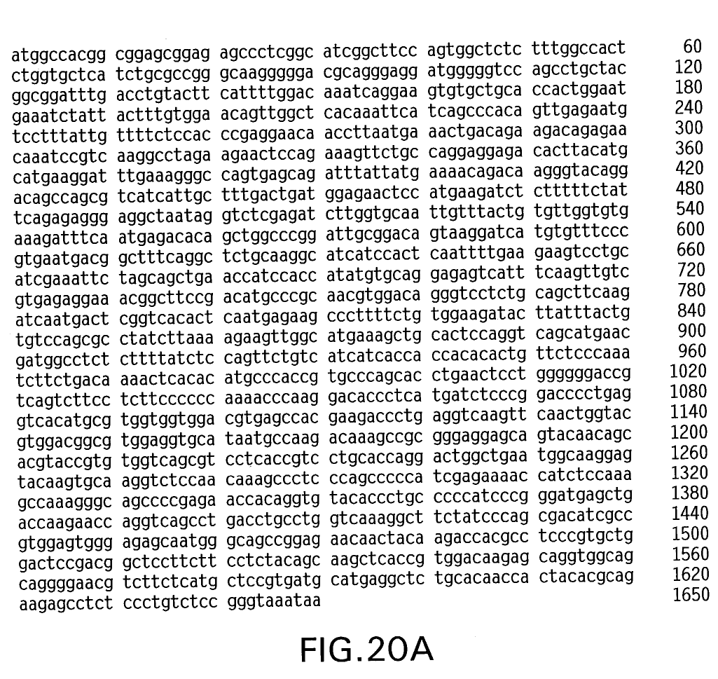

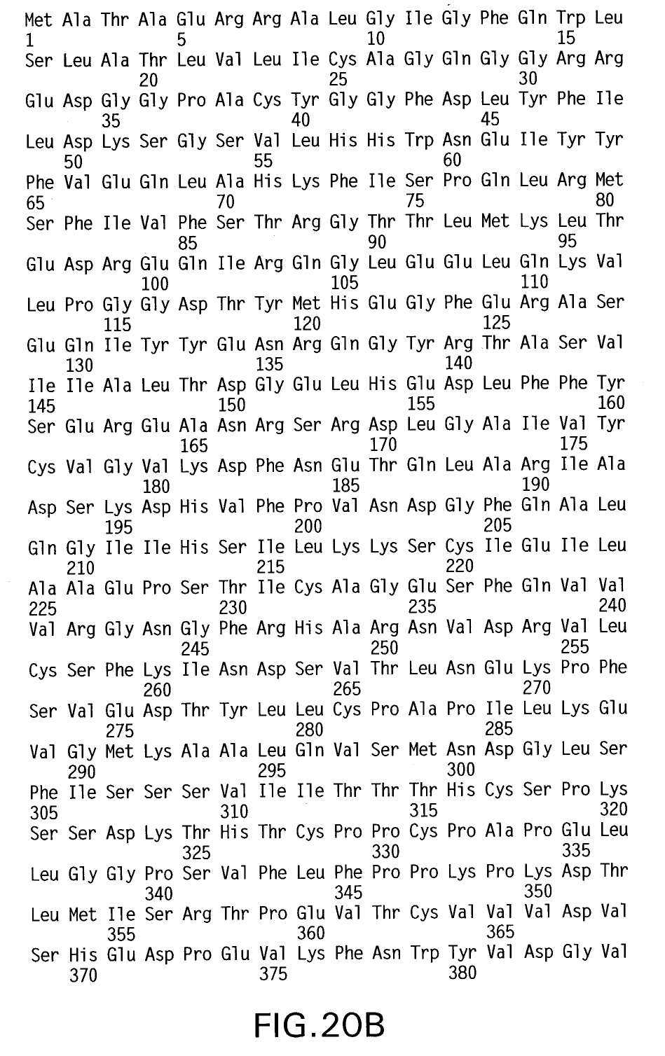

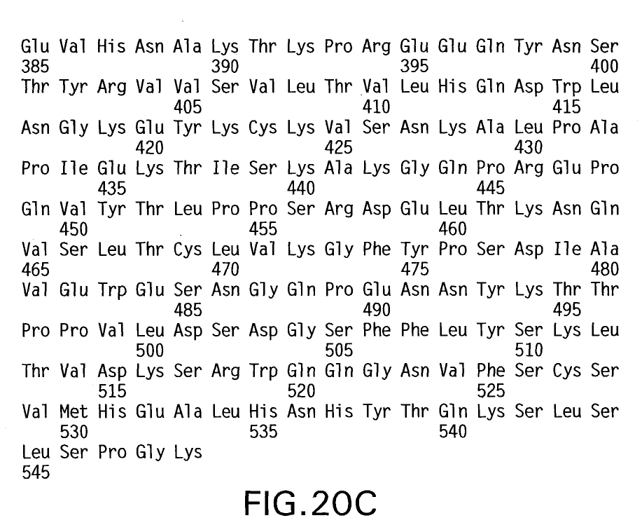

pLKTOK129: The amino terminus of the mature form of the pLKTOK129 protein comprises the extracellular region of human T197, minus the sequence of the two amino acids closest to the transmembrane region for the reasons presented above. This protein is identical to pLKTOK127 with the exception that it has a wild type Fc region of IgG1. The carboxy terminus of the pLKTOK129 protein comprises the wild type Fc sequence of human IgG1 that allows for interaction with Fc receptors. The immature form of the pLKTOK129 protein contains a human T197 signal peptide sequence. The amino acid sequence of the pLKTOK129 protein is shown in FIG. 20 (SEQ ID NO:16); the cysteine residue presented in bold represents the wild type residue that has been mutated to serine in the pLKTOK125 and pLKTOK126 proteins, and the LLGG residues presented in bold represent the wild type residues of the Fc sequences that have been mutated in the pLKTOK125 and pLKTOK126 proteins as described above. The nucleotide sequence encoding the pLKTOK125 protein is shown in FIG. 20 (SEQ ID NO:15).

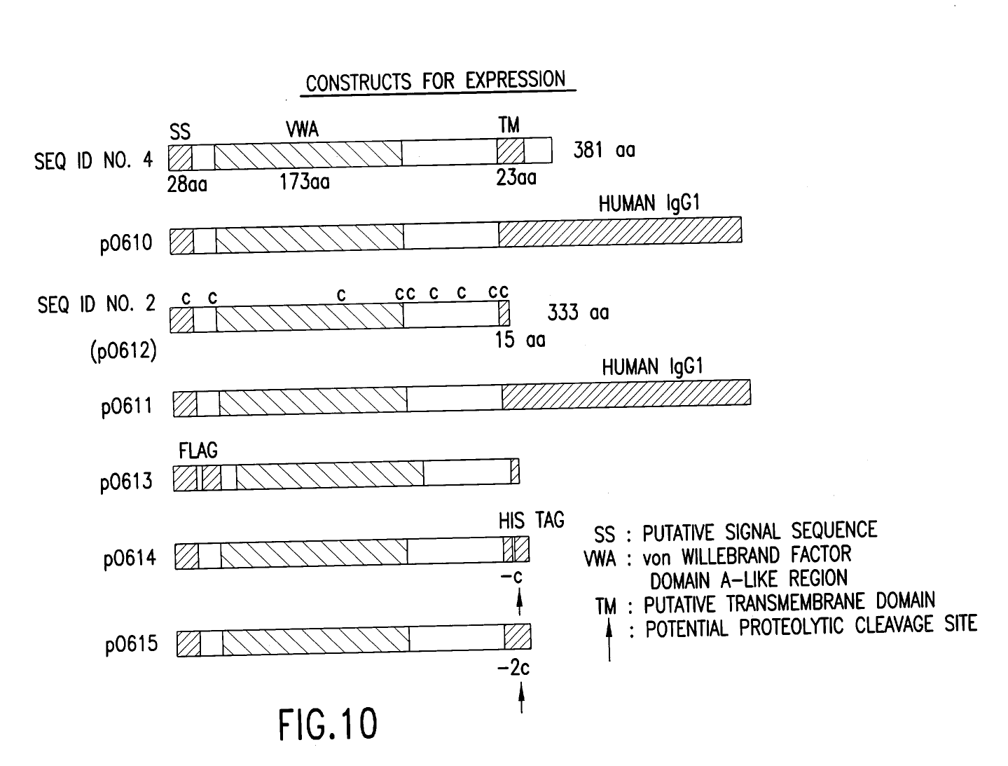

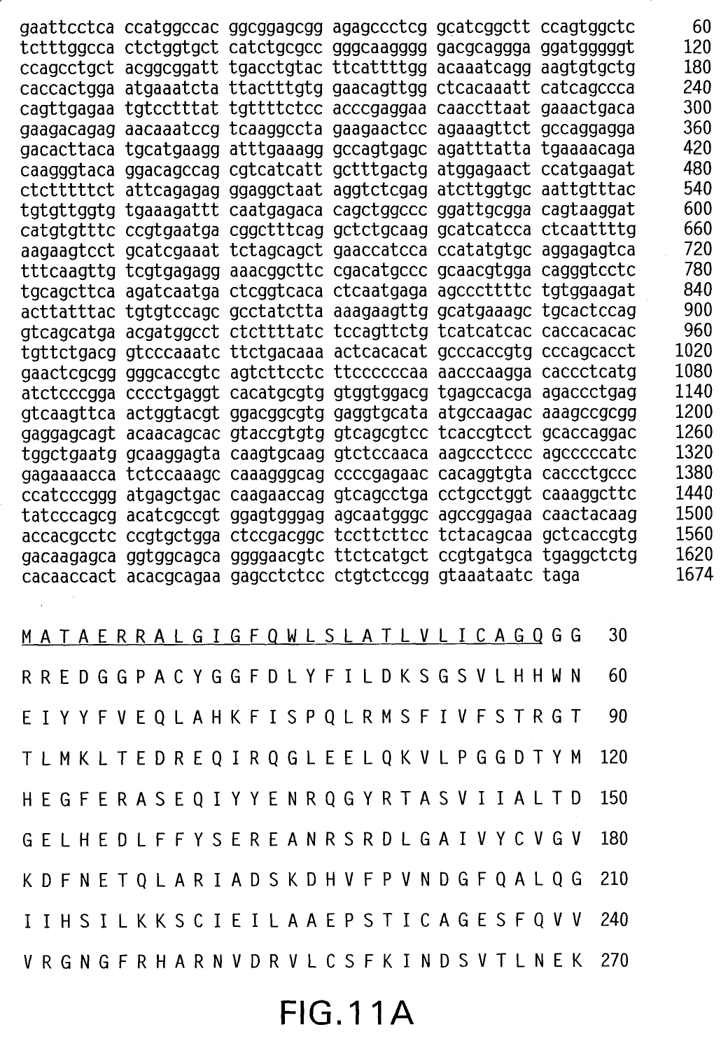

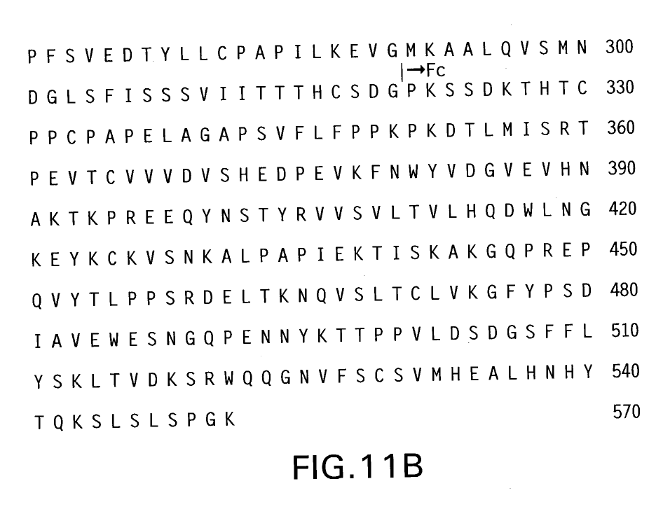

pO610: The amino terminus of the mature form of the pO610 protein comprises the extracellular region of human TANGO 197. In particular, the pO610 polypeptide comprises a human form of the mouse TANGO 197 described above. The carboxy terminus of the pO610 protein comprises the Fc sequence of human IgG1 with mutations at positions 239 (a leucine to alanine mutation relative to wild type) and 241 (a glycine to alanine mutation relative to wild type). The immature form of the pO610 protein further comprises a human TANGO 197 signal peptide (underlined in FIG. 11). The Fc portion begins at amino acid residue 321. A diagrammatic depiction of the pO610 protein is shown in FIG. 10. The amino acid sequence of the pO610 protein is shown in FIG. 10 (SEQ ID NO:18); the nucleotide sequence encoding the pO610 protein is shown in FIG. 10 (SEQ ID NO:17), with the coding region beginning at nucleotide 13 and ending at nucleotide 1666; the first six nucleotides (GAATTC) is an EcoRI restriction site and the final six nucleotides (TCTAGA) is an XbaI restriction site.

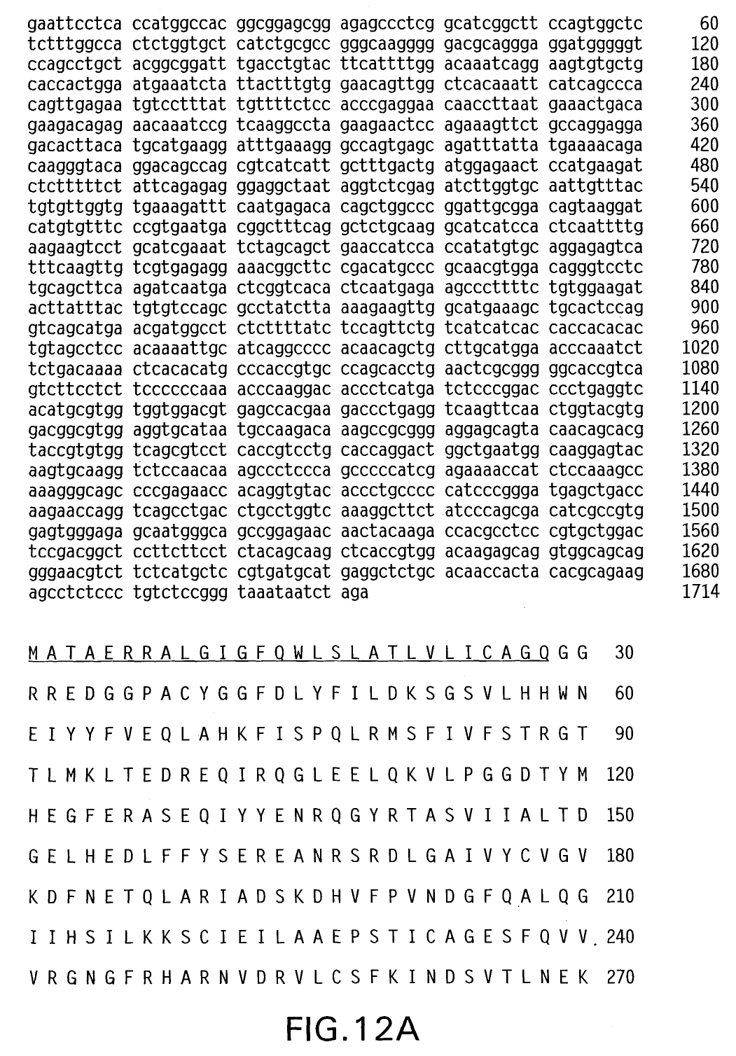

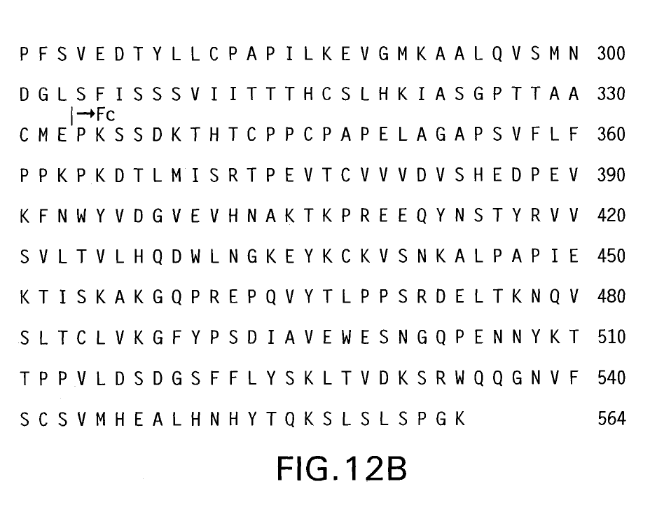

pO611: The amino terminus of the mature form of the pO611 protein comprises the mature form of human TANGO 197. The carboxy terminus of the pO611 protein comprises the Fc sequence of human IgG1 with mutations at positions 252 (a leucine to alanine mutation relative to wild type) and 254 (a glycine to alanine mutation relative to wild type). The immature form of the pO611 protein further comprises a human TANGO 197 signal peptide (underlined in FIG. 12). A diagrammatic depiction of the pO611 protein is shown in FIG. 10. The amino acid sequence of the pO611 protein is shown in FIG. 12 (SEQ ID NO:20); the nucleotide sequence encoding the pO611 protein is shown in FIG. 12 (SEQ ID NO:19), with the coding region beginning at nucleotide 13 and ending at nucleotide 1706; the first six nucleotides (GAATTC) is an EcoRI restriction site and the final six nucleotides (TCTAGA) is an XbaI restriction site.

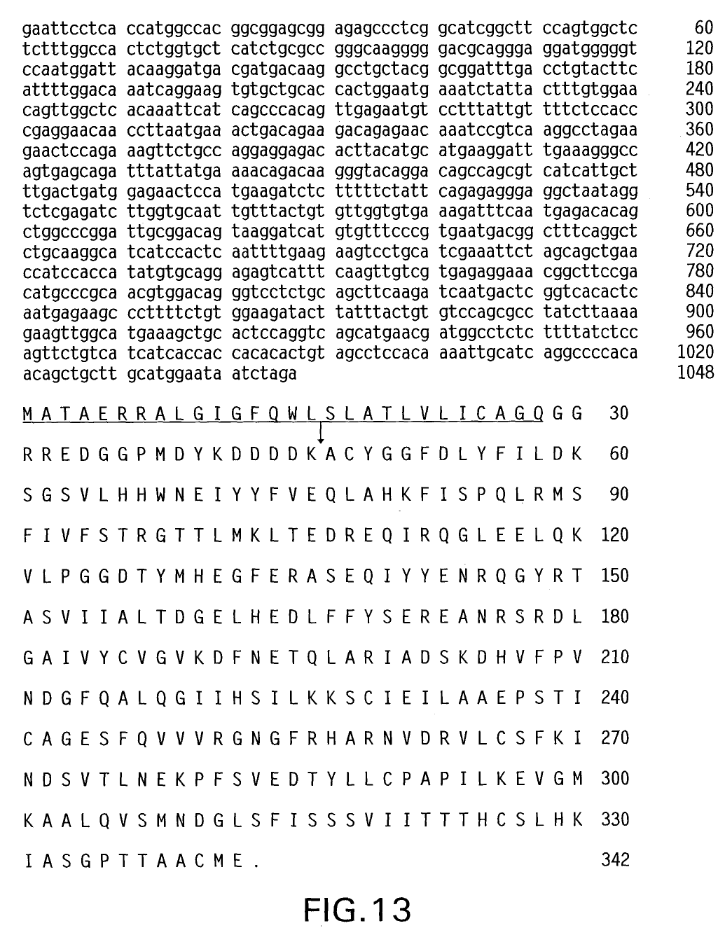

pO613: The mature form of the pO613 protein comprises human TANGO 197 with a FLAG sequence inserted almost immediately after the signal peptide cleavage site (bold in FIG. 13; arrow in the figure depicts putative proteolytic cleavage site). The immature form of the pO613 protein further comprises a human TANGO 197 signal peptide (underlined in FIG. 13). A diagrammatic depiction of the pO613 protein is shown in FIG. 10. The amino acid sequence of the pO613 protein is shown in FIG. 13 (SEQ ID NO:22); the nucleotide sequence encoding the pO611 protein is shown in FIG. 13 (SEQ ID NO:21), with the coding region beginning at nucleotide 13 and ending at nucleotide 1039; the first six nucleotides (GAATTC) is an EcoRI restriction site and the final six nucleotides (TCTAGA) is an XbaI restriction site.

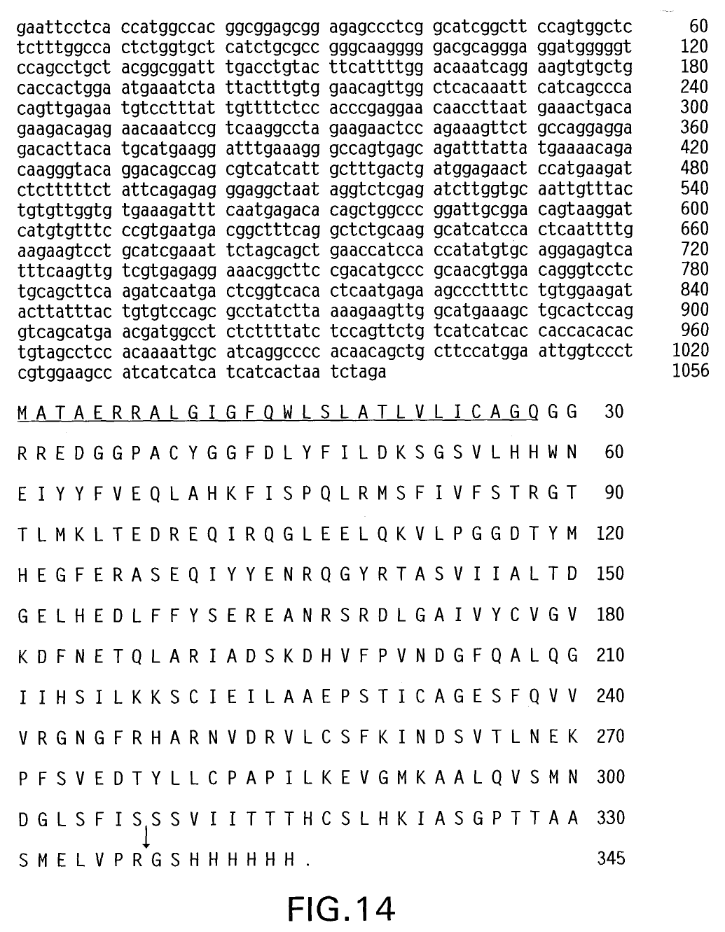

pO614: The amino terminus of the mature form of the pO614 protein comprises human TANGO 197 with the final cysteine (position 331) converted to a serine residue (bold in FIG. 14), followed by a thrombin cleavage site (LVPRGS) then HHHHHH, i.e., a HIS tag (underlined). The immature form of the pO614 protein further comprises the twenty eight amino acid human TANGO 197 signal peptide (underlined). A diagrammatic depiction of the pO614 protein is shown in FIG. 10. The amino acid sequence of the pO614 protein is shown in FIG. 14 (SEQ ID NO: 24); the nucleotide sequence encoding the pO614 protein is shown in FIG. 14 (SEQ ID NO:23), with the coding region beginning at nucleotide 13 and ending at nucleotide 1048; the first six nucleotides (GAATTC) is an EcoRI restriction site and the final six nucleotides (TCTAGA) is an XbaI restriction site.

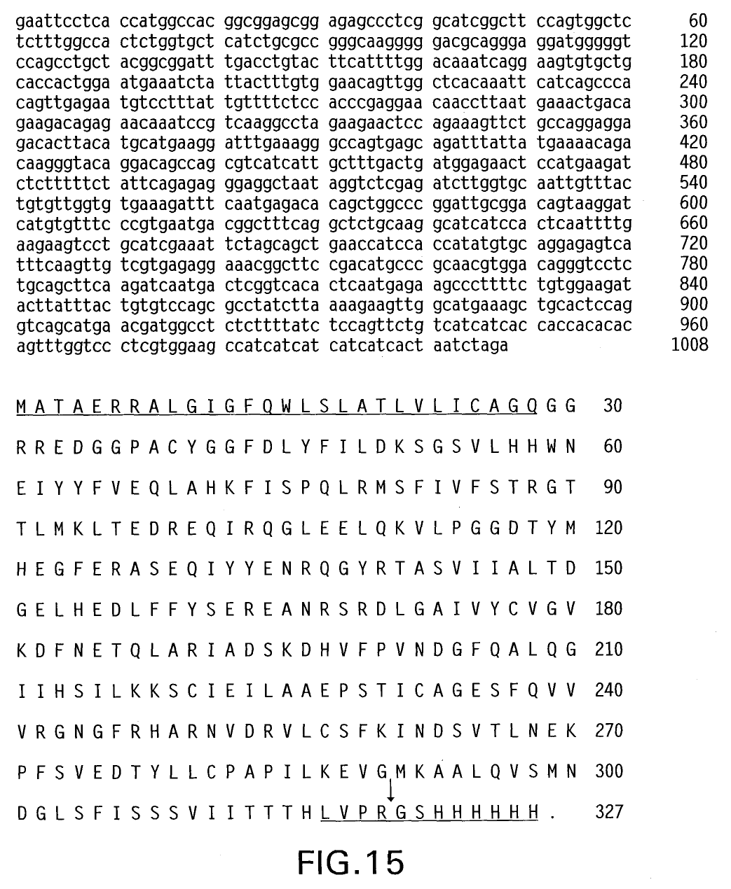

pO615: The amino terminus of the mature form of the pO615 protein comprises human TANGO 197 with the final seventeen amino acids (including the final two cysteines) removed. At the carboxy terminus of the pO615 protein, a thrombin cleavage sequences (SLVPRGS) followed by a HHHHHH, i.e., a His tag (underlined in FIG. 15) has been added. The extracellular domain deletion removes two of the cysteine residues of the wild type sequence. The immature form of the pO615 protein further comprises a human TANGO 197 signal peptide (underlined in FIG. 15). A diagrammatic depiction of the pO615 protein is shown in FIG. 10. The amino acid sequence of the pO615 protein is shown in FIG. 15 (SEQ ID NO: 26); the nucleotide sequence encoding the pO615 protein is shown in FIG. 15 (SEQ ID NO:25), with the coding region beginning at nucleotide 13 and ending at nucleotide 1000; the first six nucleotides (GAATTC) is an EcoRI restriction site and the final six nucleotides (TCTAGA) is an XbaI restriction site.

DNA constructs encoding the TANGO 197 fusion proteins pLKTOK125, pLKTOK126, pLKTOK127 and pLKTOK129 were created by PCR assembly of a series of three PCR reactions with the end result being rapid combination of two DNA fragments into a single construct, as described below. This scheme corresponds to a simplified method of procedure described in Antibody Engineering, Chapter 7, Bending, M. M. & Jones, S. T.; McCafferty et al., eds., Oxford University Press, Oxford, UK, pp. 147-168. The primers described as part of the constuctions are listed in Table 2, below.

pLKTOK125 DNA: The first reaction for pLKTOK125 was a standard 30 cycle PCR to produce two fragments, one from human splenic cDNA using the primers pTANGO 197Fa to TANGO 197Fb (fragment 1) and the second from pLKTOK56 (see U.S. patent application filed Oct. 19, 2001, internal reference number MP12001-244P1(M)) using the primers pTANGO 197Fc to pCHhum2 (fragment 2). The template, pLKTOK56, is an expression vector that contains the sequence of human IgG1 with the Fc receptor mutated from the wild-type (LLGG) to the mutant LAGA. The two fragments were gel purified and combined in equal molar ratios for assembly through 8 cycles of 94° C. for 1.5 min and 72° C. for 2.5 min with a 30 second ramping time between each. The material from this reaction was used as a template to amplify the combined sequence using the primers pTANGO 197Fa and pCHhum2. The primers TANGO 197Fb and pTANGO 197Fc contain 24 overlapping bases of complementary sequence and the DNA sequences for the mutated cystine to serine. The primer pTANGO 197Fa contains the cloning site EcoRI within its 5′ sequence (along with a Kozak sequence) and the primer pCHhum2 contains a stop codon followed by the cloning site XbaI. The amplified fragment (1674 bp) was TOPO cloned (Invitrogen, Carlsbad, Calif.) and sequenced to select for the desired clone. The nucleotide sequence of the amplified fragment is shown in FIG. 17A (SEQ ID NO:9). The sequence encoding pLKTOK125 begins at position 1 and ends at position 1647 of the nucleotide sequenced depicted in FIG. 17A. The EcoRI-XbaI fragment of the nucleotide sequence was subcloned into the expression vector pLKTOK4 (see U.S. patent application filed Oct. 19, 2001, internal reference number MP12001-244P1(M)).

pLKTOK126 DNA: As TANGO 197 contains a MfeI restriction enzyme site that would interfere with the cloning into pLKTOK55 see U.S. patent application filed Oct. 19, 2001, internal reference number MP12001-244P1(M)) (to acquire the Ig Leader) pLKTOK126 was created in three sections. The first two sections for pLKTOK126 were created using a standard 30 cycle PCR using human splenic cDNA as the template and primers pTANGO 197Fde to TANGO 197Fe mutates internal MfeI site)(section 1) and primers pTANGO 197Fg to pTANGO 197b (section 2). Section 3 used pLKTOK56 as the template with the primers pTANGO 197Fc to pCHhum2. The pTANGO 197Fe primer contains an MfeI site which allows the insert for pLKTOK126 to be functionally cloned into the expression vector pLKTOK55 and thereby acquire the human Ig leader (see U.S. patent application filed Oct. 19, 2001, internal reference number MP12001-244P1(M)). The three fragments were gel purified and combined in equal molar ratios for assembly through 8 cycles of 94° C. for 1.5 min and 72° C. for 2.5 min with a 30 second ramping time between each. The material from this reaction was used as a template to amplify the combined cDNA using the primers pTANGO 197Fd to pCHhum2. The amplified fragment was TOPO cloned (Invitrogen, Carlsbad, Calif.) cloned and sequenced to select for the desired clone. This sequence was subcloned into pLKTOK55 as an MfeI-XbaI fragment which replaces the human IgG1 (in pLKTOK55) with the TANGO 197-Fc construct but keeps the human Ig leader sequence. This sequence was subcloned into pLKTOK55 to introduce the human Ig leader sequence. The nucleotide sequence of the resulting construct is shown in FIG. 18A (SEQ. ID NO:11). The sequence encoding pLKTOK126 begins at position 1 and ends at position 1620 of the nucleotide sequence depicted in FIG. 18A. The XbaI fragment of the sequence was subcloned into the expression vector pLKTOK4.

pLKTOK127 DNA: The first reaction for pLKTOK127 was a standard 30 cycle PCR to produce two fragments, one from human splenic cDNA using the primers pTANGO 197Fa to TANGO 197Fh (fragment 1) and the second from pLKTOK56 (see U.S. patent application filed Oct. 19, 2001, internal reference number MP12001-244P1(M)) using the primers pTANGO 197Fj to pCHhum2 (fragment 2). The two fragments were gel purified and combined in equal molar ratios for assembly through 8 cycles of 94° C. for 1.5 min and 72° C. for 2.5 min with a 30 second ramping time between each. The material from this reaction was used as template to amplify the combined sequence using the primers pTANGO 197Fa and pCHhum2. The primer TANGO 197Fh contains the wild-type cysteine. The amplified fragment was TA cloned and sequenced to select for the desired clone. The nucleotide sequence of the amplified fragment is shown in FIG. 19A (SEQ ID NO:13). The sequence encoding pLKTOK127 begins at position 1 and ends at position 1647 of the nucleotide sequence depicted in FIG. 19A. The EcoRI-XbaI fragment of the nucleotide sequence was subcloned into the expression vector pLKTOK4.

pLKTOK129 DNA: This construct was made in an identical fashion tas pLKTOK127 with the exception that the template for the constant region was pLKTOK55 which contains the sequence of wild-type human IgG1. The first reaction for pLKTOK129 was a standard 30 cycle PCR to produce two fragments, one from human splenic cDNA using the primers pTANGO 197Fa to TANGO 197Fh (fragment 1) and the second from pLKTOK55 (see U.S. patent application filed Oct. 19, 2001, internal reference number MPI2001-244P1(M)) using the primers pTANGO 197Fj to pCHhum2 (fragment 2). The two fragments were gel purified and combined in equal molar ratios for assembly through 8 cycles of 94° C. for 1.5 min and 72° C. for 2.5 min with a 30 second ramping time between each. The material from this reaction was used as a template to amplify the combined sequence using the primers pTANGO 197Fa and pCHhum2. The primer TANGO 197Fh contains the wild-type cysteine. The amplified fragment was TA cloned and sequenced to select for the desired clone. The nucleotide sequence of the amplified fragment is shown in FIG. 20A (SEQ ID NO:15). The sequence encoding pLKTOK129 begins at position 1 and ends at position 1647 of the nucleotide sequence depicted in FIG. 20A. The EcoRI-XbaI fragment of the nucleotide sequence was subcloned into the expression vector pLKTOK4.

pO610 DNA: This plasmid was constructed in two stages. In the first stage, Tango 197 coding sequences and IgG1 coding sequences were separately amplified using Tango 197 and IgG1 coding plasmids (Arhob17d11 and TOK82) by utilizing partially overlapping primers. One fragment was generated by primer pairs 106/108, the other, by primer pairs 107/83. Primers 107 and 108 were partially overlapping and served to bring the Tango 197 and IgG1 encoding fragments together by PCR-ramping (8 cycles of 94 C for 1.5 min and 68 C for 2.5 min with a 30 second ramping time between each). For pO610-pO615, all PCR-reactions with the exception of “PCR-ramping” were done following the Stratagene Pro-Star protocol with 30 cycles (instead of the suggested 40 cycles).

pO611DNA: This plasmid similarly was constructed in two stages. In the first stage, Tango 197 coding sequences and IgG1 coding sequences were separately amplified using Tango 197 and IgG1 coding plasmids (Arhob17d11 and TOK82), utilising partially overlapping primers. One fragment was generated by primer pairss 106/117, the other, by primer pairs 116/83. Primers 116 and 117 were partially overlapping and served to bring the Tango 197 and IgG1 encoding fragments together by PCR-ramping (8 cycles of 94 C for 1.5 min and 68 C for 2.5 min with a 30 second ramping time between each).

pO613 DNA: was constructed using a similar strategy. pO612 was used as a template to generate two fragments using the primer sets 106/114 and 113/115 (113 and 114 partially overlapping each other). The two fragments were brought together by PCR-ramping and fused fragment amplified by using primers 106/115.

pO612, pO614, pO615 DNA: The plasmids were made using a 1-step PCR reaction from the Tango 197 plasmid (Arhob17d11). Pro-Star Ultra HF PCR-kit from Stratagene (La Jolla, Calif.; cat # 600166) was used to amplify fragments using the Tango 197 (Arhob17d11) plasmid as template.

Following amplification the pO610-pO615 fragments were purified from an agarose gel, cut with EcoRI (EcoRI recognition sequence is before the Kozak sequence and the start codon ATG) and XbaI (after the stop codon TAA), both sites introduced by primers. The cut fragments were then ligated to an expression vector (TOK10C) for transfection into 293 cells and protein expression.

TABLE 2

| | |
|---|---|
| pTANGO 197Fa: | |
| | 5′ CCGGAATTCCTCACCATGGCCACGGCGGAGCGGAGAGCCCTCGGCATCGGCTTC 3′ |
| pTANGO 197Fb: | |
| | 5′ GAAGATTTGGGAGAAGAGTGTGTGGTGGTGATGATGACAGAACTGGAGATA 3′ |
| pTANGO 197Fc: | |
| | 5′ CACTCTTCTCCCAAATCTTCTGACAAAACTCACACATGCC 3′ |
| pTANGO 197Fd: | |
| | 5′ TTACCCAATTGTGTCCTGTCCGGGGGACGCAGGGAGGATGGGGGTCCAGCCTGC 3′ |
| pTANGO 197Fe: | |
| | 5′ CACAGTAAACAATAGCACCAAGATCTCGAGACCTATTAGCCTCCCTC 3′ |
| pTANGO 197Fg: | |
| | 5′ GGTCTCGAGATCTTGGTGCTATTGTTTACTGTGTTGGTGTGAAAGATTTCA 3′ |
| pTANGO 197Fh: | |
| | 5′ GAAGATTTGGGAGAACAGTGTGTGGTGGTGATGATGACAGAACTGGAGATA 3′ |
| pTANGO 197Fj: | |
| | 5′ CACTGTTCTCCCAAATCTTCTGACAAAACTCACACATGCC 3′ |
| pCHhum2: | 5′ TGCTCTAGATTATTTACCCGGAGACAGGGAGAGGCTC 3′ |

Primers for pO610:

| | |
|---|---|
| primer 106: | 5′GTGCCCGGAATTCCTCACCATGGCCACGGCGGAGCGGAGAGCC 3′ |
| primer 107: | 5′CACTGTTCTGACGGTCCCAAATCTTCTGACAAAACTCAC 3′ |
| primer 108: | 5′GTCAGAAGATTTGGGACCGTCAGAACAGTGTGTGGTGG 3′ |
| primer 83: | 5′GGATTGCTCTAGATTATTTACCCGGAGACAGGGAG 3′ |

Primers for pO611:

| | |
|---|---|
| primer #106: | 5′ GTGCCCGGAATTCCTCACCATGGCCACGGCGGAGCGGAGAGCC 3′ |
| primer #116: | 5′ GCTGCTTGCATGGAACCCAAATCTTCTGACAAAACTCAC 3′ |
| primer #117: | 5′ GTCAGAAGATTTGGGTTCCATGCAAGCAGCTGTTGTGG 3′ |
| primer #83: | 5′GGATTGCTCTAGATTATTTACCCGGAGACAGGGAG 3′ |

Primers for pO612:

| | |
|---|---|
| primer #106: | 5′ GTGCCCGGAATTCCTCACCATGGCCACGGCGGAGCGGAGAGCC 3′ |
| primer #115: | 5′ GGATTGCTCTAGATTATTCCATGCAAGCAGCTGTTG 3′ |

Primers for pO613:

| | |
|---|---|
| primer #106: | 5′ GTGCCCGGAATTCCTCACCATGGCCACGGCGGAGCGGAGAGCC 3′ |
| primer #114: | 5′ CGTCATCCTTGTAATCCATTGGACCCCCATCCTCCCTGCGTC 3′ |
| primer #113: | 5′ ATGGATTACAAGGATGACGATGACAAGGCCTGCTACGGCGGATTTG 3′ |
| primer #115: | 5′ GGATTGCTCTAGATTATTATTCCATGCAAGCAGCTGTTG 3′ |

Primers for pO614:

| | |
|---|---|
| primer #106: | 5′ GTGCCCGGAATTCCTCACCATGGCCACGGCGGAGCGGAGAGCC 3′ |
| | 5′GATTGCTCTAGATTAGTGATGATGATGATGATGGCTTCCACGAGGGACCAAT |
| | TCCATGGAAGCAGCTGTTGTGGGGCCTG 3′ |

Primers for pO615:

| | |
|---|---|
| primer #106: | 5′ GTGCCCGGAATTCCTCACCATGGCCACGGCGGAGCGGAGAGCC 3′ |
| primer #125: | 5′GGATTGCTCTAGATTAGTGATGATGATGATGATGGCTTCCACGAGGGACCAA |
| | ACTGTGTGTGGTGGTGATGATGACAG 3′ |

### EXAMPLE

### T197 Fusion Protein Expression

This example describes the expression of TANGO 197 fusion proteins. In particular, pO610 plasmid, as described above, was expressed, and pO610 fusion polypeptide was made and secreted from the expressing cells.

The TANGO fusion polypeptide-encoding construct was transiently transfected into HEK 293T cells in 150 mM plates using Lipofectamine 2000 (GIBCO/BRL) according to the manufacturer's protocol.

72 hours post-transfection, the serum-free conditioned media (OptiMEM, Gibco/BRL) were harvested, spun, filtered and stored at 4° C. The cells were refed with medium and a second harvest carried out 72 hours later as above.

Isolation of the fusion protein was performed with a one step purification scheme utilizing the affinity of the Fc region of human IgG1 to Protein A. The conditioned media (adjusted to 2.15 M NaCl/JT Baker V24621) was passed over a 10×100 mm column packed with POROS A Protein A coupled resin available from Applied Biosystems. The column was then washed with PBS, pH 7.4 and eluted with 100 mM glycine, pH 3.0. A constant flow rate of 4 ml/min was maintained throughout the procedure and 2 mL fractions were collected.

All fractions were immediately measured. The fractions containing significant fusion protein as judged by absorbance at 280 nm were pooled, neutralized with 3.0M Tris-HCl pH 8.0 (50 uL/mL) and dialyzed in 10,000 Mw (cut off) dialysis tubing against 5L PBS, pH 7.4 at 4° C. and with constant stirring. The buffered exchanged material had glycerol added to 10% (JT Baker T38B08) and then sterile filtered with 0.2 mm filter unit (Millipore Steri-flip), aliquoted and frozen at −80° C.

Protein concentration was determined using a Bradford kit according to manufacturers instructions (Biorad). Reduced sample SDS-PAGE and Western blots revealed a single major immunoreactive band at a MW of approximately 75 kDa and non-reduced sample immunoreactive bands >75 kDa. MW estimates on SDS PAGE and Western blot are relative to standards (Invitrogen/Mark 12 stds for SDS PAGE and Multi-Mark pre-stained standards for the Western blot) The overall yield was determined to be 10 mg/L.

Deposit of Clones

A clone containing a cDNA molecules encoding TANGO 197 was deposited with the American Type Culture Collection (Manassas, Va.) as composite deposits.

A clone encoding TANGO 197 was deposited on Nov. 20, 1998 with the American Type Culture Collection under Accession Number ATCC® 98999, (also referred to herein as mix EpDHMix1) from which each clone comprising a particular cDNA clone is obtainable. This deposit is a mixture of five strains, each carrying one recombinant plasmid harboring a particular cDNA clone. To distinguish the strains and isolate a strain harboring a particular cDNA clone, one can first streak out an aliquot of the mixture to single colonies on nutrient medium (e.g., LB plates) supplemented with 100 μg/ml ampicillin, grow single colonies, and then extract the plasmid DNA using a standard minipreparation procedure. Next, one can digest a sample of the DNA minipreparation with a combination of the restriction enzymes Sal I and Not I and resolve the resultant products on a 0.8% agarose gel using standard DNA electrophoresis conditions. The digest will liberate fragments as follows:

TANGO 197 (EpDH213) 2.3 kb and 3.0 kb

A clone containing a cDNA molecule encoding human TANGO 216 (clone EpT216), was deposited with the American Type Culture Collection (Manassas, Va.) on Mar. 26, 1999 as Accession Number 207176, as part of a composite deposit representing a mixture of five strains, each carrying one recombinant plasmid harboring a particular cDNA clone.

To distinguish the strains and isolate a strain harboring a particular cDNA clone, an aliquot of the mixture can be streaked out to single colonies on nutrient medium (e.g., LB plates) supplemented with 100 μg/ml ampicillin, single colonies grown, and then plasmid DNA extracted using a standard minipreparation procedure. Next, a sample of the DNA minipreparation can be digested with a combination of the restriction enzymes Sal I and Not I and the resultant products resolved on a 0.8% agarose gel using standard DNA electrophoresis conditions. The digest liberates fragments as follows:

TANGO 216 (EpT216): 4.4 kb

All publications, patents and patent applications mentioned in this specification are herein incorporated by reference into the specification to the same extent as if each individual publication, patent or patent application was specifically and individually indicated to be incorporated herein by reference.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A method for preventing a symptom of anthrax in a subject thought to be at risk for exposure to Bacillus anthracis, comprising: administering to the subject a pharmaceutically effective amount of a fusion polypeptide so that, if the subject is exposed to Bacillus anthracis, a symptom of said exposure is prevented, wherein said fusion polypeptide comprises a von Willebrand factor A-like domain (vWF) amino acid sequence and an amino acid sequence heterologous to said vWF.

2. A method for preventing a symptom of anthrax in a subject suspected of having been exposed to Bacillus anthracis, comprising: administering to the subject a pharmaceutically effective amount of a fusion polypeptide so that, if the subject has been exposed to Bacillus anthracis, a symptom of said exposure is prevented, wherein said fusion polypeptide comprises a von Willebrand factor A-like domain (vWF) amino acid sequence and an amino acid sequence heterologous to said vWF.

3. A method for ameliorating a symptom of anthrax in a subject in need of said amelioration, comprising: administering to the subject a pharmaceutically effective amount of a fusion polypeptide so that a symptom of anthrax is ameliorated, wherein said fusion polypeptide comprises a von Willebrand factor A-like domain (vWF) amino acid sequence and an amino acid sequence heterologous to said vWF.

4. The method of claim 1, wherein the vWF is a TANGO 197 vWF as depicted in SEQ ID NO:2.

5. The method of claim 4, wherein the TANGO 197 vWF comprises amino acid residues 44-215 of the amino acid sequence depicted in SEQ ID NO:2.

6. The method of claim 5, wherein the fusion polypeptide comprises amino acid residues 29-316 of the amino acid sequence depicted in SEQ ID NO:2.

7. The method of claim 6, wherein the fusion polypeptide comprises amino acid residues 29-318 of the amino acid sequence depicted in SEQ ID NO:2.

8. The method of claim 7, wherein the fusion polypeptide comprises amino acid residues 29-333 of the amino acid sequence depicted in SEQ ID NO:2.

9. The method of claim 6, wherein the fusion polypeptide comprises amino acid residues 29-316 of the amino acid sequence depicted in SEQ ID NO:2, except that the amino acid sequence differs from that of amino acid residues 29-316 of SEQ ID NO:2 in that a cysteine residue of the amino acid sequence has been converted to another amino acid residue.

10. The method of claim 9, wherein the converted amino acid residue is not within the vWF amino acid sequence.

11. The method of claim 9, wherein the fusion polypeptide comprises amino acid residues 29-318 of the amino acid sequence depicted in SEQ ID NO:2, except that the amino acid sequence differs from that of amino acid residues 29-318 of SEQ ID NO:2 in that a cysteine residue of the amino acid sequence has been converted to another amino acid residue.

12. The method of claim 11, wherein the converted amino acid residue is not within the vWF amino acid sequence.

13. The method of claim 11, wherein the fusion polypeptide comprises amino acid residues 29-333 of the amino acid sequence depicted in SEQ ID NO:2, except that the amino acid sequence differs from that of amino acid residues 29-333 of SEQ ID NO:2 in that a cysteine residue of the amino acid sequence has been converted to another amino acid residue.

14. The method of claim 13, wherein the converted amino acid residue is not within the vWF amino acid sequence.

15. The method of claim 1, wherein the heterologous amino acid sequence comprises a human immunoglobulin constant region.

16. The method of claim 15, wherein the immunoglobulin region is a human IgG1 constant region.

17. The method of claim 16, wherein the IgG1 constant region does not bind Fc receptor.

18. The method of claim 17, wherein the IgG1 constant region does not initiate an ADCC reaction.

19. The method of claim 18, wherein the IgG1 constant region does not bind Fc receptor.

20. The method of claim 1, wherein the heterologous amino acid sequence comprises a FLAG or a His tag sequence.

21. The method of claim 20, wherein the heterologous amino acid sequence further comprises an amino acid sequence containing a proteolytic cleavage site.

22. The method of claim 1, wherein the fusion polyeptide comprises amino acid residues 29-549 of pLKTOK125 (SEQ ID NO:10), 29-540 of pLKTOK126 (SEQ ID NO:12), 29-549 of pLKTOK127 (SEQ ID NO:14), 29-549 of pLKTOK129 (SEQ ID NO:16), 29-551 of pO610 (SEQ ID NO:18), 29-564 of pO611 (SEQ ID NO:20), 29-342 of pO613 (SEQ ID NO:22), 29-345 of pO614 (SEQ ID NO:24), or 29-327 of pO615 (SEQ ID NO:26).

23. The method of claim 1, wherein the symptom is a symptom of cutaneous anthrax.

24. The method of claim 1, wherein the symptom is a symptom of inhalation anthrax.

25. A fusion polypeptide comprising a von Willebrand factor A-like domain (vWF) amino acid sequence and an amino acid sequence heterologous to said vWF.

26. The polypeptide of claim 25, wherein the vWF is a TANGO 197 vWF as depicted in SEQ ID NO:2.

27. The polypeptide of claim 26, wherein the TANGO 197 vWF comprises amino acid residues 44-215 of the amino acid sequence depicted in SEQ ID NO:2.

28. The polypeptide of claim 27, wherein the fusion polypeptide comprises amino acid residues 29-316 of the amino acid sequence depicted in SEQ ID NO:2.

29. The polypeptide of claim 28, wherein the fusion polypeptide comprises amino acid residues 29-318 of the amino acid sequence depicted in SEQ ID NO:2.

30. The polypeptide of claim 29, wherein the fusion polypeptide comprises amino acid residues 29-333 of the amino acid sequence depicted in SEQ ID NO:2.

31. The polypeptide of claim 28, wherein the fusion polypeptide comprises amino acid residues 29-316 of the amino acid sequence depicted in SEQ ID NO:2, except that the amino acid sequence differs from that of amino acid residues 29-316 of SEQ ID NO:2 in that a cysteine residue of the amino acid sequence has been converted to another amino acid residue.

32. The polypeptide of claim 31, wherein the converted amino acid residue is not within the vWF amino acid sequence.

33. The polypeptide of claim 31, wherein the fusion polypeptide comprises amino acid residues 29-318 of the amino acid sequence depicted in SEQ ID NO:2, except that the amino acid sequence differs from that of amino acid residues 29-318 of SEQ ID NO:2 in that a cysteine residue of the amino acid sequence has been converted to another amino acid residue.

34. The polypeptide of claim 11, wherein the converted amino acid residue is not within the vWF amino acid sequence.

35. The polypeptide of claim 33, wherein the fusion polypeptide comprises amino acid residues 29-333 of the amino acid sequence depicted in SEQ ID NO:2, except that the amino acid sequence differs from that of amino acid residues 29-333 of SEQ ID NO:2 in that a cysteine residue of the amino acid sequence has been converted to another amino acid residue.

36. The polypeptide of claim 33, wherein the converted amino acid residue is not within the vWF amino acid sequence.

37. The polypeptide of claim 25, wherein the heterologous amino acid sequence comprises a human immunoglobulin constant region.

38. The polypeptide of claim 37, wherein the human immunoglobulin region is a human IgG1 constant region.

39. The polypeptide of claim 38, wherein the IgG1 constant region does not bind Fc receptor.

40. The polypeptide of claim 37, wherein the IgG1 constant region does not initiate an ADCC reaction.

41. The polypeptide of claim 40, wherein the IgG1 constant region does not bind Fc receptor.

42. The polypeptide of claim 25, wherein the heterologous amino acid sequence comprises a FLAG or a His tag sequence.

43. The polypeptide of claim 42, wherein the heterologous amino acid sequence comprises an amino acid sequence containing a proteolytic cleavage site.

44. The polypeptide of claim 25, wherein the fusion polyeptide comprises amino acid residues 29-549 of pLKTOK125 (SEQ ID NO:10), 29-540 of pLKTOK126 (SEQ ID NO:12), 29-549 of pLKTOK127 (SEQ ID NO:14), 29-549 of pLKTOK129 (SEQ ID NO:16), 29-551 of pO610 (SEQ ID NO:18), 29-564 of pO611 (SEQ ID NO:20), 29-342 of pO613 (SEQ ID NO:22), 29-345 of pO614 (SEQ ID NO:24), or 29-327 of pO615 (SEQ ID NO:26).

45. The polypeptide of claim 44, wherein the fusion polypeptide comprises the amino acid sequence of pLKTOK125 (SEQ ID NO:10), pLKTOK126 (SEQ ID NO:12), pLKTOK127 (SEQ ID NO:14), pLKTOK129 (SEQ ID NO:16), pO610 (SEQ ID NO:18), pO611 (SEQ ID NO:20), pO613 (SEQ ID NO:22), pO614 (SEQ ID NO:24), or pO615 (SEQ ID NO:26).

46. A pharmaceutical composition comprising a fusion polypeptide comprising a von Willebrand factor A-like domain (vWF) amino acid sequence and an amino acid sequence heterologous to said vWF.

47. The pharmaceutical composition of claim 46, wherein the vWF is a TANGO 197 vWF as depicted in SEQ ID NO:2.

48. The pharmaceutical composition of claim 47, wherein the TANGO 197 vWF comprises amino acid residues 44-215 of the amino acid sequence depicted in SEQ ID NO:2.

49. The pharmaceutical composition of claim 48, wherein the fusion polypeptide comprises amino acid residues 29-316 of the amino acid sequence depicted in SEQ ID NO:2.

50. The pharmaceutical composition of claim 49, wherein the fusion polypeptide comprises amino acid residues 29-318 of the amino acid sequence depicted in SEQ ID NO:2.

51. The pharmaceutical composition of claim 50, wherein the fusion polypeptide comprises amino acid residues 29-333 of the amino acid sequence depicted in SEQ ID NO:2.

52. The pharmaceutical composition of claim 48, wherein the fusion polypeptide comprises amino acid residues 29-316 of the amino acid sequence depicted in SEQ ID NO:2, except that the amino acid sequence differs from that of amino acid residues 29-316 of SEQ ID NO:2 in that a cysteine residue of the amino acid sequence has been converted to another amino acid residue.

53. The pharmaceutical composition of claim 52, wherein the converted amino acid residue is not within the vWF amino acid sequence.

54. The pharmaceutical composition of claim 52, wherein the fusion polypeptide comprises amino acid residues 29-318 of the amino acid sequence depicted in SEQ ID NO:2, except that the amino acid sequence differs from that of amino acid residues 29-318 of SEQ ID NO:2 in that a cysteine residue of the amino acid sequence has been converted to another amino acid residue.

55. The pharmaceutical composition of claim 54, wherein the converted amino acid residue is not within the vWF amino acid sequence.

56. The pharmaceutical composition of claim 54, wherein the fusion polypeptide comprises amino acid residues 29-333 of the amino acid sequence depicted in SEQ ID NO:2, except that the amino acid sequence differs from that of amino acid residues 29-333 of SEQ ID NO:2 in that a cysteine residue of the amino acid sequence has been converted to another amino acid residue.

57. The pharmaceutical composition of claim 56, wherein the converted amino acid residue is not within the vWF amino acid sequence.

58. The pharmaceutical composition of claim 46, wherein the heterologous amino acid sequence comprises a human immunoglobulin constant region.

59. The pharmaceutical composition of claim 58, wherein the human immunoglobulin region is a human IgG1 constant region.

60. The pharmaceutical composition of claim 59, wherein the IgG1 constant region does not bind Fc receptor.

61. The pharmaceutical composition of claim 59, wherein the IgG1 constant region does not initiate an ADCC reaction.

62. The pharmaceutical composition of claim 61, wherein the IgG1 constant region does not bind Fc receptor.

63. The pharmaceutical composition of claim 46, wherein the heterologous amino acid sequence comprises a FLAG or a His tag sequence.

64. The pharmaceutical composition of claim 63, wherein the heterologous amino acid sequence further comprises an amino acid sequence containing a proteolytic cleavage site.

65. The pharmaceutical composition of claim 46, wherein the fusion polyeptide comprises amino acid residues 29-549 of pLKTOK125 (SEQ ID NO:10), 29-540 of pLKTOK126 (SEQ ID NO:12), 29-549 of pLKTOK127 (SEQ ID NO:14), 29-549 of pLKTOK129 (SEQ ID NO:16), 29-551 of pO610 (SEQ ID NO:18), 29-564 of pO611 (SEQ ID NO:20), 29-342 of pO613 (SEQ ID NO:22), 29-345 of pO614 (SEQ ID NO:24), or 29-327 of pO615 (SEQ ID NO:26).
